SEQUENCE LISTING (24 sequences)

SEQ ID NO: 1 - DNA, 18 bp, Artificial Sequence
  Synthetic construct
ggcagcaagaggatcagg

SEQ ID NO: 2 - DNA, 18 bp, Artificial Sequence
  Synthetic construct
ccgtagagatcggcgaca

SEQ ID NO: 3 - DNA, 20 bp, Artificial Sequence
  Synthetic construct
gggcaacacctctacaagga

SEQ ID NO: 4 - DNA, 19 bp, Artificial Sequence
  Sythetic construct
cttgggtctgggtttcctc

SEQ ID NO: 5 - DNA, 20 bp, Artificial Sequence
  Synthetic construct
acgtaccggccactatcact

SEQ ID NO: 6 - DNA, 20 bp, Artificial Sequence
  Synthetic construct
ctcgagtctgcacatcagga

SEQ ID NO: 7 - DNA, 19 bp, Artificial Sequence
  Synthetic construct
agccacatcgctcagacac

SEQ ID NO: 8 - DNA, 19 bp, Artificial Sequence
  Synthetic construct
gcccaatacgaccaaatcc

SEQ ID NO: 9 - DNA, 19 bp, Artificial Sequence
  Synthetic construct
gagagatcccactacgaaa

SEQ ID NO: 10 - DNA, 19 bp, Artificial Sequence
  Synthetic construct
gtgggagcgcgtgatgaac

SEQ ID NO: 11 - RNA, 21 nt, Artificial Sequence
  Synthetic construct
uggaauucucgggugccaagg

SEQ ID NO: 12 - RNA, 26 nt, Artificial Sequence
  Synthetic construct
guucagaguucuacaguccgacgauc

SEQ ID NO: 13 - DNA, 21 bp, Artificial Sequence
  Synthetic construct
tcgtatgccgtcttctgcttg

SEQ ID NO: 14 - DNA, 24 bp, Artificial Sequence
  Synthetic cosntruct
atctcgtatgccgtcttctgcttg

SEQ ID NO: 15 - DNA, 32 bp, Artificial Sequence
  Synthetic construct
cgacaggttcagagttctacagtccgacgatc

SEQ ID NO: 16 - DNA, 21 bp, Artificial Sequence
  Synthetic construct
tggaattctcgggtgccaagg

SEQ ID NO: 17 - DNA, 50 bp, Artificial Sequence
  Synthetic construct
aaaaaaaaaaaaaaaaaaaaaaaaaaaaaaaaaaaaaaaaaaaaaaaaaa

SEQ ID NO: 18 - DNA, 50 bp, Artificial Sequence
  Synthetic construct
tttttttttttttttttttttttttttttttttttttttttttttttttt

SEQ ID NO: 19 - DNA, 25 bp, Artificial Sequence
  Synthetic construct
gtgacacaactgtgtgattaaaagg

SEQ ID NO: 20 - DNA, 26 bp, Artificial Sequence
  Synthetic construct
tgatttttattattttggtaatggtg

SEQ ID NO: 21 - DNA, 20 bp, Artificial Sequence
  Synthetic construct
tctctggctgagggatgact

SEQ ID NO: 22 - DNA, 24 bp, Artificial Sequence
  Synthetic construct
ttttaaggccaaggaattaagtga

SEQ ID NO: 23 - DNA, 29 bp, Artificial Sequence
  Synthetic construct
ctcctcttcaccaggtatcctgatgtgca

SEQ ID NO: 24 - DNA, 29 bp, Artificial Sequence
  Synthetic construct
ttcgagcagctcaaccgcaacttcagcaa

(19) United States
(12) Patent Application Publication
(10) Pub. No.: US 2019/0209618 A1
(43) Pub. Date: Jul. 11, 2019

(54) METHODS AND COMPOSITIONS FOR EXPANSION OF HEMATOPOIETIC STEM AND/OR PROGENITOR CELLS EMPLOYING A CYTOCHROME P450 1B1 (CYP1B1) INHIBITOR OR A MUSASHI-2 (MSI2) ACTIVATOR

(71) Applicant: McMaster University, Hamilton (CA)

(72) Inventors: Kristin Hope, Burlington (CA); Stefan Rentas, Cambridge (CA)

(21) Appl. No.: 16/096,429

(22) PCT Filed: Apr. 26, 2017
(86) PCT No.: PCT/CA2017/050511

§ 371 Date: Oct. 25, 2018

Related U.S. Application Data
(60) Provisional application No. 62/327,649, filed on Apr. 26, 2016.

(51) Int. Cl. A61K 35/28; A61K 31/7048; A61K 31/37; A61K 31/09; A61P 35/02; A61K 31/352; C07D 311/18; C07D 311/32; C07D 473/00; C12N 15/113; A61P 37/00; A61P 7/00

(52) U.S. Cl. CPC A61K 35/28; A61K 45/06; A61K 31/37; A61K 31/09; A61P 35/02; A61K 31/352; C07D 311/18; C07D 311/32; C07D 473/00; C12N 15/1137; A61P 37/00; A61P 7/00; C12N 2310/14; C12N 2310/531; A61K 31/7048

(57) ABSTRACT

A method of increasing the self-renewal and/or expansion of hematopoietic stem and/or progenitor cells (HSPCs) is described. Inhibiting the activity and/or expression of cytochrome P450 1B1 (CYP1B1) and/or increasing the expression or activity of Musashi-2 (MSI2) increases the expansion of HSPCs. The HSPCs may be cultured in the presence of a CYP1B1 inhibitor and/or a MSI2 activator. Optionally, the cells may be expanded ex vivo and transplanted into a subject in need thereof.

### RELATED APPLICATIONS

This application claims the benefit of priority to U.S. Provisional application No. 62/327,649 filed Apr. 26, 2016, the contents of which are incorporated herein by reference in their entirety.

### FIELD OF INVENTION

The present invention pertains to the field of hematopoietic stem cells and more specifically to methods and compositions useful for expanding hematopoietic stem and/or progenitor cells.

### BACKGROUND OF THE INVENTION

Although umbilical cord blood (CB)-derived hematopoietic stem cells (HSCs) are essential in many life-saving regenerative therapies, their limited number in CB units has significantly restricted their clinical use and the subsequent advantages they provide during transplantation (Miller et al. 2013). Select small molecules that enhance hematopoietic stem and/or progenitor cell (HSPC) expansion in culture have been identified (Boitano et al. 2010; Fares et al., 2014) however, in many cases their mechanisms of action or the nature of the pathways they impinge on are poorly understood. A greater understanding of the molecular pathways that underpin the unique human HSC self-renewal program will facilitate the development of targeted strategies that expand these critical cell types for regenerative therapies. Whereas transcription factor networks have been shown to influence the self-renewal and lineage decisions of human HSCs (Novershtern et al., 2011; Laurenti et al., 2013), the post-transcriptional mechanisms guiding HSC fate have not been closely investigated.

### SUMMARY OF THE INVENTION

In one aspect, the inventors have shown that overexpression of the RNA-binding protein (RBP) Musashi-2 (MSI2) induces multiple pro-self-renewal phenotypes. Overexpression of MSI2 resulted in a 17-fold increase in short-term repopulating cells and a net 23-fold ex vivo expansion of long-term repopulating HSCs. The inventors have also determined that MSI2 directly attenuates aryl hydrocarbon receptor (AHR) signaling through post-transcriptional downregulation of canonical AHR pathway components in cord blood (CB) HSPCs by performing a global analysis of MSI2-RNA interactions. This provides new insights into RBP-controlled RNA networks that underlie the self-renewal process. These networks may be manipulated to enhance the regenerative potential of human HSCs. Examination of MSI2 OE-induced differentially expressed genes found Cytochrome P450 1B1 Oxidase (CYP1B1), an effector of AHR signaling (Mimura et al., 2013) amongst the most repressed. As shown in the Examples, a number of inhibitors of CYP1B1, including but not limited to (E)-2,3′,4,5′-Tetramethoxystilbene (TMS), Chrysin, Narigenin and Acacetin as well as siRNA that targets CYP1B1 expression have also been demonstrated to promote the self-renewal and expansion of hematopoietic stem and progenitor cell cultures.

Accordingly, in one aspect there is provided a method of increasing the self-renewal and/or expansion of hematopoietic stem and/or progenitor cells (HSPCs) by increasing the expression of MSI2 in a population of one or more HSPCs.

In another aspect, there is provided a method of increasing the renewal and/or expansion of HSPCs by inhibiting the activity and/or expression of cytochrome P450 1B1 (CYP1B1) in a population of one or more HSPCs.

In one embodiment, the HSPCs are CD34+ cells. Optionally, the HSPCs are from cord blood, umbilical cord, mobilized peripheral blood or bone marrow. In one embodiment, the HSPCs are in vivo, ex vivo or in vitro.

In one embodiment, the methods described herein comprise contacting HSPCs with a CYP1B1 inhibitor. Examples of CYP1B1 inhibitors include, but are not limited to stilbenoids, flavonoids, coumarins or alkaloids. In one embodiment, the stilbenoid is 2,3′,4,5′-TMS. Additional examples of CYP1B1 inhibitors include antisense and/or siRNA molecules that target CYP1B1 expression, such as shRNA.

In one embodiment, CYP1B1 inhibitor is a flavonoid selected from 3,5,7-Trihydroxy-2-(4-hydroxyphenyl)-4H-chromen-4-one (Kaempferol), 5,7-Dihydroxy-2-phenyl-4H-chromen-4-one (Chrysin), 5,7-Dihydroxy-2-(4-hydroxyphenyl)chroman-4-one (Naringenin), 3,6,7-Trihydroxy-2-(4-hydroxy-3-methoxyphenyl)-4H-chromen-4-one (Isohamnetin), 3′,5-Dihydroxy-4′,6,7-trimethoxyflavone (Eupatorin), 2-(3,4-Dihydroxyphenyl)-5,7-dihydroxy-4-chromenone (Luteolin), 5,7-dihydroxy-2-(4-methoxyphenyl)-4H-1-benzopyran-4-one (Acacetin), 2-(3,4-Dimethoxyphenyl)-5,6,7,8-tetramethoxychromen-4-one (Nobiletin), 2-(3,4-dihydroxyphenyl)-3,5,7-trihydroxy-4H-chromen-4-one (Quercetin), 5,7-Dihydroxy-2-(4-hydroxyphenyl)-4H-1-benzopyran-4-one (Apigenin), 5,7-Dihydroxy-3-(4-hydroxyphenyl)chromen-4-one (Genistein), 7-Hydroxy-3-(4-hydroxyphenyl) chromen-4-one (Daidzein) and 5-hydroxy-3-(4-hydroxyphenyl)-7-methoxychromen-4-one (Prunetin).

In one embodiment, the CYP1B1 inhibitor is a coumarin such as is 6-,7-dihydroxycoumarin or 5-methoxypsoralen (bergapten).

In one embodiment, the CYP1B1 inhibitor is an alkaloid such as evodiamine.

In one embodiment, the CYP1B1 inhibitor is chrysin, Naringenin, Bergapten, 6-,7-dihydroxycoumarin or Quercetin.

In one embodiment, the CYP1B1 inhibitor is evodiamine, 6,7-dihydroxycoumarin, bergapten, chrysin, naringenin, isohamnetin, eupatorin, liteloin, acacetin, kaempferol, nobiletin, quercetin, apigenin, genistein, daidzein, prunetin or TMS.

In one embodiment, the CYP1B1 inhibitor has an IC₅₀ of less than 10 μM.

In one embodiment, the CYP1B1 inhibitor increases the frequency of CD34+ cells in the population of cells relative to a control population of cells. In one embodiment, the CYP1B1 inhibitor increases the number of CD34+ cells in the population of cells relative to a control population of cells.

In another embodiment, the methods described herein comprise contacting HSPCs with a MSI2 activator.

In yet another embodiment, the methods described herein comprise contacting HSPCs with a CYP1B1 inhibitor and an agent that induces hematopoiesis, optionally SR1 or a pyrimidoindole derivative.

In another aspect, there is provided a population of HSPCs expanded using the methods described herein. In one embodiment, the expanded HSPCs are for administration or use in the treatment of a subject in need thereof. The HSPCs may be autologous HSPCs or allogenic HSPCs. In one embodiment, the subject has a hematopoietic disorder, malignancy, autoimmune disease and/or immunodeficiency. In one embodiment, the subject has a thalassemia or anemia. In one embodiment, the subject has a cancer such as blood cancer or bone marrow cancer. In one embodiment the subject has lymphoma, multiple myeloma or leukemia.

In one embodiment, there is provided a method for increasing the self-renewal and/or expansion of HSPCs in a subject in need thereof. In one embodiment, the method comprises administering to the subject a CYP1B1 inhibitor and/or an agent that increases the activity and/or expression of MSI2 as described herein. In one embodiment, the CYP1B1 inhibitor and/or agent that increases the activity and/or expression of MSI2 is administered before, concurrently or after the transplantation of autologous or allogenic HSPCs to the subject.

Also provided is a CYP1B1 inhibitor and/or an agent that increases the activity and/or expression of MSI2 as described herein for use in increasing the self-renewal and/or expansion of HSPCs in a subject in need thereof. In one embodiment, the CYP1B1 inhibitor and/or agent that increases the activity and/or expression of MSI2 is administered before, concurrently or after the transplantation of autologous or allogenic HSPCs cells to the subject.

Optionally, the methods and/or uses described include the administration or use of an agent that induces hematopoiesis such as SR1 or a pyrimidoindole derivative, concurrently or after the transplantation of autologous or allogenic HSPCs cells to the subject.

In one embodiment, there is provided a composition comprising a CYP1B1 inhibitor as described herein and an agent that induces hematopoiesis such as SR1 or a pyrimidoindole derivative. In one embodiment, there is provided a composition comprising an agent that increases the activity and/or expression of MSI2 as described herein and an agent that induces hematopoiesis such as SR1 or a pyrimidoindole derivative. In one embodiment, the composition is a growth media, such as a growth media suitable for HSPCs. In one embodiment, the composition is a pharmaceutical composition suitable for administration to a subject in need thereof.

In another aspect, there is provided a composition comprising one or more HSPCs and a CYP1B1 inhibitor. In a further aspect, there is provided a composition comprising one or more HSPCs and a MSI2 activator. In one embodiment, the composition is a cell culture. In another embodiment, the composition is a pharmaceutical composition, optionally for administration or use in the treatment of a subject in need thereof. For example, in one embodiment, the composition is for use in the treatment of a hematopoietic disorder, malignancy, autoimmune disease and/or immunodeficiency that would benefit from the transplantation of HSPCs.

In another aspect, there is provided a method of producing a culture of leukemic cells. In one embodiment, the method comprises inhibiting the activity and/or expression of cytochrome P450 1B1 (CYP1B1) in a population of one or more leukemic cells. In another embodiment, the method comprises increasing the activity and/or expression of Mushashi-2 (MSI2) in a population of one or more leukemic cells. In one embodiment, the cells are in vitro. In one embodiment, the cells are cultured in the presence of a CYP1B1 inhibitor. In one embodiment, the leukemic cells are acute myeloid leukemia (AML) cells.

In another aspect, there is provided a method for identifying agents with activity against leukemic cells that may be useful as chemotherapeutic agents. In one embodiment, the method comprises providing a culture of leukemic cells produced using the methods described herein, contacting the culture of leukemic cells with a test agent and determining whether the test agent inhibits the proliferation of leukemic cells in the cell culture relative to a control.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1: MSI2 OE enhances in vitro CB progenitor activity and increases numbers of STRCs. a, CFU output from transduced Lin⁻ CB (n=9 control and 10 MSI2 OE cultures from 5 experiments). b, CFU-GEMM secondary CFU replating potential (n=24 control and 30 MSI2 OE from 2 experiments) and images of primary GEMMs (scale bar 200 μm). c, Number of secondary colonies per replated CFU-GEMM from b. d, CD34 expression in STRCs prior to transplant (n=3 experiments). e, Human chimerism at 3 weeks in mice transplanted with varying doses of transduced STRCs. Dashed line indicates engraftment cutoff (n=3 experiments). f, STRC frequency as determined by LDA from e. Dashed lines indicate 95% C.I. Data shown as mean±SEM. *p<0.05, **p<0.01, ***p<0.001.

FIG. 2: MSI2 OE expands LT-HSCs with ex vivo culture. a, Transduced CD34⁺CD133⁺ cells after one week of culture (n=4 experiments, unpaired t-test). b-d, CD45⁺GFP⁺ engraftment from mice receiving the highest two cell doses for D3 and D10 (n=8 mice for both conditions) and the highest three doses for D10 secondary mice (n=6 control and 9 MSI2 OE mice, Mann-Whitney test). e, Myelo-lymphopoiesis in D10 secondary mice. f, Multi-lineage LT-HSC frequency in BM cells of D10 primary mice. Dashed lines indicate 95% C.I. g, Numbers of GFP⁺ HSCs as evaluated by LDA. h, Cumulative fold change in MSI2 OE-transduced HSCs. Data shown as mean±SEM. *p<0.05, **p<0.01.

FIG. 3: MSI2 OE in human HSPCs attenuates AHR signaling. a, Predicted AHR targets compared by GSEA to genes downregulated with MSI2 OE. b, GSEA of SR1 downregulated gene sets and MSI2 OE downregulated genes. c, Log fold-change of MSI2 OE and SR1 GSEA leading edge genes. d, Percentage of gene overlap between UM171-, SR1-treated and MSI2 OE downregulated gene sets and AHR ChIP-seq-identified targets. e, Number of CFU-GEMMs generated from transduced cells grown in CFU medium containing FICZ or DMSO (n=3 experiments). f, CFU-GEMMs from e replated into CFU assays containing FICZ or DMSO (n=30 control and 29 MSI2 OE per condition). Data are presented as mean±SEM. **p<0.01.

FIG. 4: MSI2 OE post-transcriptionally downregulates AHR pathway components. a, Overlap between MSI2 target genes from separate CLIP-seq experiments. b, Statistically significant overlap (p<0.0001, hypergeometric test) of clusters between the replicates. c, Percent of CLIP-seq clusters in different genic regions. d, Consensus motifs within MSI2 clusters in different genic regions. P-values presented for the top 40% of clusters. e, CLIP-seq reads (replicate 1: blue, replicate 2: green) and clusters (purple) mapped to the 3′UTR of CYP1B1. Matches to the GUAG motif shown in black. f and g, Immunofluorescent images of HSP90 and CYP1B1 3 days after transduction and summary of fold-change in HSP90 and CYP1B1 protein and transcript levels with MSI2 OE at 3 and 7 days post transduction (dotted line indicates no change, n=3 experiments). h, HSPC marker expression by CD34⁺ cells treated with TMS for 10 days. i, Absolute CD34⁺ cell number with TMS (n=4 experiments). Data are presented as mean±SEM. **p<0.01.

FIG. 5: MSI2 is highly expressed in human hematopoietic stem and progenitor cell populations. a, Schematic of the human hematopoietic hierarchy showing key primitive cell populations and simplified surface marker expression. b, qRT-PCR analysis of MS/1 and MSI2 expression in Lin⁻ CB cell populations (n=3 independent Lin− CB samples). c, Gating strategy used to sort sub-fractions of Lin⁻ CB HSPCs for MSI2 qRT-PCR expression analysis (n=2 independent pooled Lin⁻ CB samples). d, MSI2 expression across the human hematopoietic hierarchy. Each circle represents an independent gene expression dataset curated by HemaExplorer. e, Intracellular flow cytometry analysis of MSI2 protein levels in Lin⁻ CB. Histograms show background staining with secondary antibody (red) and positive staining with anti-MSI2 antibody plus secondary in Lin⁻ CB (blue). MSI2 fluorescence intensity was divided into quartiles of negative (Q1), low (Q2), mid (Q3) and high (Q4) level expression. f, Plots show cell percentage within each quartile from e that are CD34⁺CD38⁻(left) and CD34⁺CD38⁺(right) (n=2 independent Lin⁻ CB samples). All data presented as mean±SEM. *p<0.05, ***p<0.001.

FIG. 6: MSI2 OE enhances in vitro culture of primitive CB cells. a, Top: Schematic of bi-directional promoter lentivirus used to overexpress MSI2. Bottom: Western blot and histogram showing intracellular flow validation of enforced MSI2 expression in 293FT cells (left) and Lin⁻ CB (right), respectively. b, Representative images of secondary CFU made from replated control and MSI2 OE CFU-GEMMs and types of colonies made, scale bar=200 μm. c, Fold change in Lin⁻ CB transduced cell number after 7 days in culture following transduction (n=5 experiments). d, 21 day growth curve of transduced Lin⁻ CB cells (n=4 experiments). e, Colony output of transduced Lin⁻ CB from day 7 cultures (n=8 cultures from 4 experiments). f, BrdU cell cycle analysis of transduced Lin⁻ CB cells from day 10 cultures (n=3 experiments). g, Ki67 cell cycle analysis of transduced Lin⁻ CB cells from day 4 cultures (n=4 experiments). h, Apoptotic and dead cells in day 7 cultures of transduced Lin⁻ CB by Annexin V staining (n=3 experiments). All data presented as mean±SEM. **p<0.01, ***p<0.001.

FIG. 7: MSI2 OE does not affect STRC lineage output and extends STRC-mediated engraftment. a, Schematic of STRC LDA experimental setup. b, Left: Gating strategy to identify engrafted GFP⁺CD45⁺ progenitor and myelo-lympho lineage positive cell types or GFP⁺CD45⁻ erythroid cells and platelets. Right: Summary of lineage output in the injected femur at 3 weeks post-transplant (n=4 control and n=18 MSI2 OE mice). MK=Megakaryocyte, E=Erythroid cells, P=Platelets. c, Representative flow plots and summary of transduced STRCs read out for human CD45⁺ engraftment at 6.5 weeks post-transplant (n=4 mice per condition). All data presented as mean±SEM.

FIG. 8: MSI2 KD impairs secondary CFU replating potential and HSC engraftment capacity. a, Left: Schematic of MSI2- and control RFP-targeted shRNA lentiviruses. Right: Confirmation of MSI2 protein knockdown (both isoforms that can be detected by western blot) in transduced NB4 cells. b, CFU production by shMSI2 and shControl transduced Lin⁻ CB (n=8 cultures from 4 experiments). c, Secondary CFU output from shMSI2 and images of representative secondary CFU (scale bar=200 μm, performed on n=4 cultures from 2 experiments). d, Fold change in transduced cell number after 7 days in culture (n=4 experiments). e, Growth curves of cultures initiated with transduced Lin⁻ CB cells (n=4 experiments). f, Experimental design to readout changes in HSC capacity with MSI2 KD. g, Left: Representative flow analysis of transduced CD34⁺CD38⁻ derived human chimerism in NSG mouse BM. Right: Shown is the ratio of the percentage of GFP⁺ cells in the CD45⁺ population post-transplant to the initial pre-transplant GFP⁺ cell percentage. Dotted line indicates the proportion of GFP⁺ cells is unchanged relative to input. (One sample t-test, no change=1, n=6 shControl and n=8 shMSI2 mice pooled from 2 experiments). h, Representative flow plots and summary of multilineage engraftment with shControl and shMSI2 (gated on GFP⁺ cells). Data presented as mean±SEM. *p<0.05, ***p<0.001.

FIG. 9: MSI2 OE confers an HSC gene expression signature. a, Genes that are up in OE (21 genes, log FC>0) and down (156 genes, log FC<0) relative to control with a FDR<0.05 were compared to MSI2 KD normalized to shControl expression data. Red circles represent 177 significantly differentially expressed genes with OE. Gray outlined circles represent random genes (number of gray circles=number of red circles). Only genes that are significantly up or down regulated in OE have anti-correlation with KD. b, Differentially expressed genes between MSI2 OE and control cells (FDR<0.05) compared to DMAP populations. Numbers beside each bar indicate the percentage of the time the observed value (set of up or downregulated genes) was better represented in that population than random values (equal number of randomly selected genes based on 1000 trials).

FIG. 10: MSI2 OE enhances HSC activity after ex vivo culture. a, Experimental layout to measure changes in HSC engraftment capacity and frequency with ex vivo culture. b, Representative flow plots of CD45⁺ GFP⁺ reconstitution from mice receiving the highest cell dose transplanted per time point. c, Multilineage engraftment of mice injected with D3 cultures. d, Proportion of the human CD45⁺ graft containing GFP⁺ cells from D3 primary mice relative to pre-transplant levels of GFP⁺ cells before expansion. Shown are mice transplanted at the two highest doses (n=8 mice for both conditions). e, Proportion of the human CD45⁺ graft containing GFP⁺ cells from D10 primary mice relative to pre-transplant levels of GFP⁺ cells after expansion. Shown are mice transplanted at the two highest doses (n=8 mice for both conditions, one sample t-test, no change=1). f, Summary of multilineage engraftment from mice in c. g, GFP mean fluorescence intensity (MFI) in D10 primary engrafted mice. Shown are mice transplanted with the highest three doses, n=11 control and 13 MSI2 mice. h, CD34 expression in GFP-high (top 60%) relative to GFP-low (bottom 40%) gated cells (set per mouse) from engrafted recipients in c. i, Number of transduced phenotyped HSCs after 7 days of culture from HSC expansion experiment described in a. Symbols represent individual mice and shaded symbols represent MSI2 OE. All data presented as mean±SEM.

FIG. 11: Predicted AHR targets and genes downregulated with SR1 and MSI2 OE are upregulated with MSI2 KD. a, Predicted AHR targets were identified with the iRegulon tool and compared with MSI2 KD normalized to shControl upregulated gene signature by GSEA. b, Heatmap of MSI2 OE and KD shared leading edge AHR target genes from GSEA. c, GSEA comparing SR1 high and low dose downregulated gene sets with the MSI2 KD upregulated gene signature. d, Heatmap shows list of shared leading edge genes identified by GSEA from MSI2 OE, KD and SR1 at varying doses. e, The percentage of downregulated genes from UM171-, SR1-treated and MSI2 OE data sets containing at least one AHR binding site 1500 bp upstream and downstream of transcription start site. Dotted line indicates the background percentage of genes with AHR binding sites. P-values were generated relative to background with Fisher's exact test.

FIG. 12: AHR antagonism with SR1 has redundant effects with MSI2 OE, and AHR activation with MSI2 OE results in a loss of HSPCs. a, Representative flow plots and summary of CD34 expression with MSI2 OE and control transduced CD34⁺CB cells grown for 10 days in medium containing SR1 or DMSO vehicle (n=3 experiments). b, Fold change in CD34 expression from results shown in a. c, Fold-increase in CYP1B1 and AHRR transcript levels with FICZ in transduced cultures (n=3 experiments). d, Transduced CD34⁺CB cells grown for 3 days in medium supplemented with FICZ and the corresponding change in CD34 expression. Each pair (DMSO and FICZ) represents a matched CB sample (n=3 experiments). e, Differences in culture CD34 levels from d. All data presented as mean±SEM. * p<0.05.

FIG. 13: MSI2 preferentially binds mature mRNA within the 3′UTR. a, Validation of the capacity of the anti-MSI2 antibody to immunoprecipitate MSI2 compared to IgG control pulldowns. b, Autoradiogram showing anti-MSI2 immunoprecipitated, MNase digested and radiolabelled RNA isolated for CLIP library construction and sequencing (red box). Low levels of MNase show a smearing pattern extending upwards from the modal weight of MSI2. c, Scatter plot of total number of uniquely mapped CLIP-seq reads for each gene, comparing both replicates. d, Heatmap indicating the number of different classes of Gencode annotated genes that contain at least one predicted MSI2 binding site. e, Consensus motifs within MSI2 clusters in the different genic regions. P-values for the most statistically significant enriched motif is presented for all overlapping clusters between replicates. f, Cumulative distribution function of mean conservation score (Phastcons) of MSI2 clusters, compared to a shuffled background control, computed for all overlapping clusters and the top 40% of overlapping clusters. P-values were obtained by a Kolgomorov-Smirnov two-tailed test comparing the distributions from actual and shuffled locations. g, Number of clusters within 200 bases of the annotated stop codon in known mRNA transcripts for all overlapping clusters between replicates and the top 40% of overlapping clusters. h, Cumulative distribution function of mean conservation score (Phastcons) of MSI2 clusters, compared to a shuffled background control, computed for overlapping clusters between the replicates and the top 40% of overlapping clusters found in different genic regions. Similarity in the 3′UTR conservation for the top 40% with the shuffled background control is likely due to MSI2 sites being small and not needing structural contexts for conservation. P-values were obtained by a Kolgomorov-Smirnov two-tailed test comparing the distributions from actual and shuffled locations. i, Genome browser views displaying CLIP-seq mapped reads from replicate 1, predicted clusters, exact matches for the GUAG sequence (black) and mammal conservation scores (PhyloP) in the 3′ UTRs for a previously predicted Msi1 target.

FIG. 14: MSI2 OE represses CYP1B1 and HSP90 3′UTR Renilla Luciferase reporter activity. a, CLIP-seq reads (replicate 1 and replicate 2) and clusters mapped to the 3′UTR of HSP90. Matches to the GUAG motif are shown in black. Mammal PhyloP score listed in last track. b and c, Representative data of mean per cell fluoresence for HSP90 and CYP1B1 protein in transduced CD34⁺CB. Protein level in cells during in vitro culture was analyzed 3 days (D3) and 7 days (D7) after transduction and sorting for GFP. Corresponding secondary alone antibody staining is shown for each experiment. Each circle represents a cell, and greater than 200 cells were analyzed per condition. d and e, Levels of renilla luciferase activity in NIH-3T3 cells co-transfected with control or MSI2 OE vectors and the CYP1B1 or HSP90 wild type or TCC mutant 3′UTR luciferase reporter (dotted line indicates no change in renilla activity; n=4 CYP1B1 and n=3 HSP90 experiments). f, Flow plots of co-transduced CD34⁺CB cells with MSI2 (GFP) and CYP1B1 (BFP) lentivirus. g, GFP⁺ BFP⁺ CFU-GEMMs generated from f were replated in to secondary CFU assays and enumerated for total number of colonies formed. A total of 24 CFU-GEMMs from MSI2-BFP and MSI2-CYP1B1 were replated (n=2 experiments). Data presented as mean±SEM. ***p<0.001, **p<0.01. h, A model for AHR pathway attenuation through MSI2 post-transcriptional processing. MSI2 mediates the post-transcriptional down regulation of HSP90 at the outset of culture and continuously represses the prominent AHR pathway effector CYP1B1 to facilitate HSPC expansion. The resultant MSI2-mediated repression of AHR signaling enforces a self-renewal program and allows HSPC expansion ex vivo.

FIG. 15: CYP1B1 knockdown increases the number of CD34 marked HSPCs. (A) Schematic of lentiviral vector used to knockdown CYP1B1 and validation of transcript level repression after transduction in NB4 cells. (B) Lin− CB cells were transduced with non-targeting control shRNA or shCYP1B1 lentivirus. CD34 marker expression was measured by flow cytometry after 7 days of in vitro culture. Data shown as mean+/−SEM from n=2 experiments performed from a single patient CB sample.

FIG. 16: Primary screen for inhibitors of Cytochrome P40 oxidases and promotion of in vitro HSPC expansion by primitive marker expression. 1000 flow sorted Lin− CD34+CB HSPCs were plated per well of a 96-well plate and test compounds were added at three doses (0.1 uM, 1.0 uM, 10 uM) in triplicate. Cell cultures were refreshed with medium every 3 days and at day 10 cells were counted and assessed for CD34 marker expression. Black-thatched bars represent the range of enhancement provided by TMS over DMSO vehicle and grey-thatched bar represent the range of improved CD34 output with SR1 over vehicle. Columns marked by “x” have a >2-fold increase compared to DMSO. Columns marked by “o” show at least a 1.5-fold increase compared to DMSO control. Trypanthrin, rutaecarpine and evodiamine are alkaloids, 6,7-dihydroxycoumarin, 7,8-dihydroxycoumarin, osthole, 6-geranyl-7-hydroxycoumarin, 6,7-dihydroxybergamottin, bergamottin, imperatorin and bergapten are coumarins, chrysin, naringenin, tangeretin, isohamnetin, eupatorin, diosmetin, luteolin, acacetin, myricetin, kaempferol, nobiletin, quercetin, apigenin, genistein, daidzein and prunetin are flavonoids and TMS is a stilbene. Data shown as mean+/−SEM from n=3 cultures.

FIG. 17: Primary screen for inhibitors of Cytochrome P40 oxidases and promotion of in vitro HSPC expansion by total number CD34 marked cells. 1000 flow sorted Lin− CD34+CB HSPCs were plated per well of a 96-well plate and test compounds were added at three doses (0.1 uM, 1.0 uM, 10 uM) in triplicate. Cell cultures were refreshed with medium every 3 days and at day 10 cells were counted and assessed for CD34 marker expression. Black-thatched bars represent the range of enhancement provided by TMS over DMSO vehicle and grey-thatched bar represent the range of improved CD34 output with SR1 over DMSO vehicle. Columns marked by “x” have a >2-fold increase compared to DMSO. Columns marked by “o” show at least a 1.5-fold increase compared to DMSO control. Evodiamine is an alkaloid, 6-,7-dihydroxycoumarin, 6-geranyl-7-hydroxycoumarin and bergapten are coumarins, naringenin, chrysin, luteolin, acacetin, kaempferol, apigenin, nobiletin, quercetin, genistein and daidzein are flavonoids, and TMS is a stilbene. Data shown as mean+/−SEM from n=3 cultures.

FIG. 18: HSPCs cultured with SR1+TMS have higher frequency and number of CD34+ marked cells in long-term cultures. (A) Percentage of CD34 marked HSPCs during in vitro culture with SR1 and TMS. (B) Total number of CD34+ cells after 17 days in culture with SR1 and TMS. Data shown as mean+1- SEM from n=3 experiments.

### DETAILED DESCRIPTION

The inventors have determined that overexpression of the RNA-binding protein (RBP) Musashi-2 (MSI2) induces multiple pro-self-renewal phenotypes. MSI2 has also been shown to attenuate aryl hydrocarbon receptor (AHR) signaling in HSPCs. Remarkably, the inventors have also shown that directly inhibiting CYP1B1 results in an increase in self-renewal and expansion of HSPCs. Previously, CY1B1 expression has been used as an indicator of AHR signaling activity however it was not know that inhibiting CYP1B1 activity could be used to expand HSPCs.

As used herein, “hematopoietic stem cell” refers to a cell that is capable of self-renewal and differentiating into all specialized cell types of the hematopoietic lineage.

As used herein, “hematopoietic progenitor cell” refers to a cell that is lineage restricted and capable of giving rise to either the myeloid or lymphoid lineage of cells.

As used herein “differentiation” refers to the process by which a less specialized cell such as a stem cell develops or matures to possess a more distinct form and function with a concomitant loss of potential. Cells that are less specialized can be differentiated into cells that are more specialized by culturing the cells under particular conditions or in specific media as known in the art.

As used herein “culturing” refers to maintaining cells in media with or without cell division or differentiation for any period of time.

As used herein, “hematopoietic lineage” refers to cells that give rise to cells typically found in the blood including hematopoietic stem cells, hemogenic precursors and mature cell types from the myeloid lineages (monocytes and macrophages, neutrophils, basophils, eosinophils, erythrocytes, megakaryocytes/platelets, dendritic cells), and lymphoid lineages (T-cells, B-cells, NK-cells).

The term “leukemia” as used herein refers to any disease involving the progressive proliferation of abnormal leukocytes found in hematopoietic tissues, other organs and usually in the blood in increased numbers. “Leukemic cells” refers to leukocytes characterized by an increased abnormal proliferation of such cells.

As used herein, “acute myelogenous leukemia” (“AML”) refers to a cancer of the myeloid line of blood cells, characterized by the rapid growth of abnormal white blood cells that accumulate in the bone marrow and interfere with the production of normal blood cells.

The term “subject” as used herein includes all members of the animal kingdom including mammals, and suitably refers to humans. Optionally, the term “subject” includes mammals that have been diagnosed with a hematopoietic disorder, malignancy, autoimmune disease and/or immunodeficiency. In one embodiment, the subject is a subject in need of a transplant of HSPCs. In one embodiment, the subject has received, is in the process of receiving, or will receive a transplant of HSPCs.

### Methods of Expanding HSPCs

In one embodiment, there is provided a method of increasing the self-renewal and/or expansion of hematopoietic stem and progenitor cells (HSPCs) by inhibiting the activity and/or expression of cytochrome P450 1B1 (CYP1B1). In one embodiment, the method comprises inhibiting CYP1B1 in a population of cells comprising one or more HSPCs.

In another embodiment, there is provided a method of increasing the self-renewal and/or expansion of HSPCs by increasing the expression or activity of Musashi-2 (MSI2) in a population of one or more HSPCs.

In one embodiment, expanding the HSPCS increases the frequency and/or number of HSPCs in the population of cells. For example, inhibiting CYP1B1 or increasing the expression or activity of MSI2 in a population of HSPCs may increase the relative number of HSPCs relative to other cell types and/or total number of HSPCs in a population of cells. In one embodiment, inhibiting CYP1B1 increases the frequency and/or number of HSPCs in a population of cells relative to a control population wherein CYP1B1 has not been inhibited. In another embodiment, increasing the expression or activity of MSI2 increases the frequency and/or number of HSPCs in a population of cells relative to a control population wherein expression or activity of MSI2 has not been increased.

In one embodiment, expanding the HSPCS increases the frequency and/or number of HSPCs in the population of cells by at least 5%, 10%, 25%, 50%, 75%, 100%, 150% or 200% relative to the control population of cells.

The methods and compositions described herein may be used to expand an initial population of one or more HSPCs. For example, in one embodiment the initial HSPCs are from cord blood, umbilical cord, mobilized peripheral blood or bone marrow.

In one embodiment, the methods described herein comprise testing a population of cells for the expression of one or more biomarkers. In one embodiment, cells are isolated from a population of cells comprising HSPCs prior to or after expanding the HSPCs using the methods described herein. In one embodiment, one or more HSPCs that express certain biomarkers are separated from a population of cells using positive or negative selection techniques. For example, in one embodiment HSPCs are separated from a sample of cord blood prior to contacting the HSPCs with a CYP1B1 inhibitor or a MSI2 activator. In one embodiment, the cells are separated using Fluorescence Activated Cell Sorting (FACS) or other techniques for separating cells based on the expression of specific markers known in the art.

In one embodiment, hematopoietic stem cells may be separated from a population of HPSCs after expansion to produce a population of isolated hematopoietic stem cells. In another embodiment, hematopoietic progenitor cells may be separated from a population of HPSCs after expansion to produce a population of isolated hematopoietic progenitor cells, optionally common myeloid progenitors or common lymphoid progenitors.

In one embodiment, biomarkers for human hematopoietic stem cells are CD34⁺, CD59⁺, CD90/Thy1⁺, CD38^{low/-}, c-Kit^{-/low}, and/or Lin⁻.

In one embodiment, biomarkers for human hematopoietic progenitor cells include CD34 and Flt-3/Flk-2. Human hematopoietic progenitor cells include two types of progenitor cells with specific lineage commitments; common myeloid progenitors (CMPs) and common lymphoid progenitors (CLPs). Biomarkers for CMPs include IL-3 R alpha. Human bone marrow CLPs are CD34⁺CD38⁺Neprilysin⁺. Human cord blood CLPs are CD34⁺CD38⁻CD7⁺.

The methods and composition described herein may be used to expand a population of one or more HSPCs in in vivo, ex vivo or in vitro. In one embodiment, the cells are from a single subject. Alternatively, the cells are from two or more subjects. In some embodiments, the HSPCs expanded according to the methods describe herein are for transplantation. Optionally, the expanded HSPCs are autologous cells or allogenic cells.

Different methods may be used to inhibit the activity and/or expression of CYP1B1 in order to expand a population of HSPCs as described herein. In a preferred embodiment, the HSPCs are contacted with a CYP1B1 inhibitor. In one embodiment, the HSPCs are contacted with two or more CYP1B1 inhibitors. In one embodiment, contacting the HSPCs with a CYP1B1 inhibitor comprises culturing the cells in the presence the CYP1B1 inhibitor, such as by adding the inhibitor to a growth medium.

A “CYP1B1 inhibitor” as used herein includes any substance that is capable of inhibiting the expression or activity of CYP1B1.

A number of different CYP1B1 inhibitors known in the art may be useful for expanding HSPCs. For example, Liu et al., 2013 (incorporated by reference in its entirety) describes different classes of CYP1B1 inhibitors suitable for use in the methods described herein including stilbenoids, flavonoids, coumarins or alkaloids. Optionally, the CYP1B1 inhibitor inhibits CYP1B1 with an 1050 of less than 20 μM, less than 10 μM, less than 5 μM, less than 1 μM, less than 0.5 μM or less than 0.1 μM.

In one embodiment, the CYP1B1 inhibitor is a stilbenoid. Examples of stilbenoids useful for inhibiting CYP1B1 include, but are not limited to, 2,4,3′,5′-Tetramethoxystilbene (2,4,3′,5′-TMS), 2,4,2′,6′-Tetramethoxystilbene (2,4,2′,6′-TMS) and (E)-2,3′,4,5′-Tetramethoxystilbene (2,3′,4,5′-TMS). In one embodiment, the stilbenoid is 2,3′,4,5′-TMS.

In one embodiment, the CYP1B1 inhibitor is a flavonoid. Examples of flavonoids useful for inhibiting CYP1B1 include, but are not limited to, 3,5,7-trihydroxyflavone, 4′-methoxy-5,7-dihydroxyflavone, 3′,4′-dimethoxy-5,7-dihydroxyflavone, chrysin, naringenin, tangeretin, isohamnetin, eupatorin, diosmetin, luteolin, acacetin, myricetin, kaempferol, nobiletin, quercetin, apigenin, genistein, daidzein and prunetin. Bioflavonoids useful for inhibiting CYP1B1 are also disclosed in Doostdar et al. (2000), hereby incorporated by reference in its entirety.

In one embodiment, the CYP1B1 inhibitor is a coumarin. Examples of coumarins useful for inhibiting CYP1B1 include, but are not limited to, furocoumarins, bergamottin, coriandrin, 6,7-dihydroxycoumarin, 7,8-dihydroxycoumarin, osthole, 6-geranyl-7-hydroxycoumarin, 6,7-dihydroxybergamottin, imperatorin and bergapten.

In one embodiment, the CYP1B1 inhibitor is an alkaloid. Examples of alkaloids useful for inhibiting CYP1B1 include, but are not limited to rutaecarpines, such as 10- and 11-methoxyrutaecarpine, Berberine, palmatine, jatrorrhizine, trypanthrin and evodiamine.

In one embodiment, the CYP1B1 inhibitor has an IC50 of less than 100 μM, less than 50 μM, less than 25 μM, less than 10 μM, less than 5 less than 1 μM, less than 0.5 μM or less than 0.05 μM. In one embodiment, the CYP1B1 inhibitor has an 1050 less than 10 μM, less than 1 or less than 0.1 μM.

Other CYP1B1 inhibitors known in the art such as synthetic molecules, natural products and/or antibodies may also be used in the methods and compositions described herein. In one embodiment, the CYP1B1 inhibitor is an antisense nucleic acid molecule, siRNA, protein, antibody (or fragment thereof), aptamer, peptibody or small molecule inhibitor. In an embodiment, the inhibitor is a blocking antibody or fragment thereof against CYP1B1.

The term “antisense nucleic acid” as used herein means a nucleic acid that is produced from a sequence that is inverted relative to its normal presentation for transcription. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added. Thus, in another embodiment, the CYP1B1 inhibitor is an siRNA molecule that decreases expression of CYP1B1. In one embodiment, the siRNA is a shRNA that inhibits the expression of CYP1B1 and has sequence identity to SEQ ID NO: 23 or 24. In one embodiment, the siRNA comprises or consists of a nucleic acid sequence with at least 80%, 85%, 90%, 95% or 100% identity to SEQ ID NO: 23 or to SEQ ID NO: 24.

The term “aptamer” as used herein refers to short strands of nucleic acids that can adopt highly specific 3-dimensional conformations. Aptamers can exhibit high binding affinity and specificity to a target molecule. These properties allow such molecules to specifically inhibit the functional activity of proteins. Thus, in another embodiment, the CYP1B1 inhibitor is an aptamer that binds and inhibits CYP1B1 activity.

The term “peptibody” as used herein refers to a recombinant protein that fuses a peptide region with the Fc region of IgG. Thus, in another embodiment, the CYP1B1 inhibitor is a CYP1B1 peptide inhibitor fused with the Fc region of IgG.

The term “antibody” as used herein is intended to include monoclonal antibodies, polyclonal antibodies, chimeric antibodies and humanized antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and domain antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

In another embodiment, there is provided a method of increasing the self-renewal and/or expansion of hematopoietic stem and progenitor cells (HSPCs) by contacting the HSPCs with a CYP1B1 inhibitor in combination with at least one additional agent. In one embodiment, the additional agent is an agent that induces hematopoiesis. In one embodiment, the agent that induces hematopoiesis is an AHR antagonist such as SR1. In another embodiment, the agent that induces hematopoiesis is a pyrimindoindole derivative. Examples of pyrimindoindole derivatives include UM171, UM125729 (UM729), UM118428 (Tranylcypromine HCl), UM121184 (2-Hydroxyxanthone), UM1211179 (Retusin 7-methyl ether), UM125454, UM121064 (3-Hydroxyflavone) and UM117304 (Pratol). Pyrimindoindole derivatives also include those described in Fares et al., 2014, which is incorporated herein by reference in its entirety.

The HSPCs may be contacted with the additional agent prior to, overlapping with, concurrently, and/or after being contacted with the CYP1B1 inhibitor. The combination of CYP1B1 inhibitor and the additional agent may act to increase the self-renewal and/or expansion of HSPCs.

Different methods may be used to increase the activity and/or expression of MSI2 in order to expand a population of HSPCs as described herein.

The term “increased expression” or “overexpression” as used herein means any form of expression of the MSI2 gene or gene product that is additional to the original wild-type MSI2 expression level.

Methods for increasing expression of genes or gene products are well known in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution, or isolated promoters may be introduced into a cell in the proper orientation and distance from a gene of interest so as to control the expression of the gene.

In one embodiment, the HSPCs are contacted with a MSI2 activator. In one embodiment, the HSPCs are contacted with two or more MSI2 activators. In one embodiment, contacting the HSPCs with a MSI2 activator comprises culturing the cells in the presence of a MSI2 activator, such as by adding the activator to a growth medium.

A “MSI2 activator” as used herein includes any substance that is capable of increasing the expression or activity of MSI2 and includes, without limitation, additional MSI2 nucleic acid or protein or fragments thereof, small molecule activators, antibodies (and fragments thereof), and other substances that can activate MSI2 expression or activity.

In one embodiment, the MSI2 activator is a MSI2 mRNA or DNA molecule or fragment thereof. In another embodiment, the MSI2 activator is a MSI protein or functional fragment thereof. In another embodiment, the MSI protein or functional fragment thereof is conjugated, directly or indirectly, to a protein transfection reagent that facilitates transfection of the protein into a HSPC. Protein transfection reagents normally form non-covalent complexes with the protein to be delivered and carry it through the plasma membrane. Examples of protein transfection reagents include, but are not limited to, cell penetrating peptides or protein transduction domains, lipids and complexes thereof. For example, Gundry et al., (2016) reports refined electroporation conditions for introducing recombinant protein into primitive human blood cells. A protein of interest may also be coupled to a transduction domain like TAT as described in Krosl et al., (2003) or to a cell penetrating peptide as described in Bolhassani et al., (2017)

In one embodiment, HSPCs may be contacted with an additional agent that induces hematopoiesis as described herein prior to, overlapping with, concurrently, and/or after being contacted with the MSI2 activator.

In one embodiment, the methods described herein also include the use of a combination of a CYP1B1 inhibitor and a MSI2 activator for increasing the self-renewal and/or expansion of HSPCs. In one embodiment, the CYP1B1 inhibitor and the MSI2 activator are for use or administration concurrently, overlapping or separately. In one embodiment, the methods described herein include contacting HSPCs with the combination of a CYP1B1 inhibitor and a MSI2 activator either at the same time or at different times. In one embodiment, the methods and/or uses described herein include contacting HSPCs with a composition comprising a CYP1B1 inhibitor and a MSI2 activator.

### Expanded HSPCs for Therapeutic Use

In one embodiment, HSPCs expanded according to the methods described herein may be useful for transplantation or use in a subject in need thereof. Optionally, the expanded HSPCs are autologous HSPCs taken from the subject and expanded ex vivo or allogenic HSPCs taken from one or more donor subjects.

In one embodiment, a CYP1B1 inhibitor and/or MSI2 activator as described herein may be used and/or administered to a subject in order to increase the self-renewal and/or expansion of HSPCs in vivo, optionally before, during or after a transplant of HSPCs.

In one embodiment, the subject has a disease or disorder that can be treated by the use or administration of HSPCs, including but not limited to a hematopoietic disorder, malignancy, autoimmune disease and/or immunodeficiency. In one embodiment, the hematopoietic disorder is thalassemia, sickle cell anemia, aplastic anemia or fanconi anemia. In one embodiment, the malignancy is a cancer of the blood or bone marrow. In one embodiment, the cancer is non-Hodgkin's lymphoma, Hodgkin's lymphoma, multiple myeloma or leukemia. In some embodiments, the subject has received chemotherapy and/or radiation. In one embodiment, the chemotherapy and/or radiation has resulted in a hematopoietic disorder in the subject.

In one embodiment, the methods and compositions described herein provide for the treatment of a subject in need thereof who would benefit from transplantation of HSPCs. The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. In one embodiment, treatment methods comprise administering to a subject a therapeutically effective amount of expanded HSPCs as described herein and optionally consists of a single administration, or alternatively comprises a series of administrations.

In one embodiment, the methods and uses described herein involve the administration or use of an effective amount of HSPCs. As used herein, the phrase “effective amount” or “therapeutically effective amount” means an amount effective, at dosages and for periods of time necessary to achieve the desired result. For example in the context of treating a hematopoietic disorder such as anemia, an effective amount is an amount that for example induces an increase in the amount of red blood cells in the blood compared to the response obtained without administration of the HSPCs. Effective amounts may vary according to factors such as the disease state, age, sex and weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

### Compositions

In one embodiment, there is provided a composition comprising one or more HSPCs and a cytochrome P450 1B1 (CYP1B1) inhibitor. Also provided is a composition comprising one or more HSPCs, a cytochrome P450 1B1 (CYP1B1) inhibitor and an agent that induces hematopoiesis, optionally SR1 or a pyrimidoindole derivative.

In another embodiment, there is provided a composition comprising one or more HSPCs and a Musashi-2 (MSI2) activator.

In one embodiment, the composition is a cell culture. Optionally, the cell culture comprises a CYP1B1 inhibitor and further comprises one or more factors that encourage the growth or expansion of HSPCs. In one embodiment, the cell culture further comprises an agent that induces hematopoiesis.

In another embodiment, the cell culture comprises a MSI2 activator and further comprises one or more factors that encourage the growth or expansion of HSPCs.

In one embodiment, the composition is a pharmaceutical composition. In one embodiment, the pharmaceutical composition comprises one or more HSPCs, a CYP1B1 inhibitor and a pharmaceutically acceptable carrier. Optionally, the pharmaceutical composition further comprises an agent that induces hematopoiesis.

In another embodiment, the pharmaceutical composition comprises one or more HSPCs, a MSI2 activator and a pharmaceutically acceptable carrier.

In one embodiment, the HSPCs may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The term “pharmaceutically acceptable” means compatible with the treatment of animals, in particular, humans. Optionally, the pharmaceutical composition is formulated for intravenous administration.

In one embodiment, the pharmaceutical compositions are useful for the treatment of a hematopoietic disorder, malignancy, autoimmune disease and/or immunodeficiency in a subject in need thereof.

### Methods of Culturing Leukemic Cells

Leukemic cells and in particular AML cells can be difficult to culture and expand in vitro. Compositions and methods described herein for expanding populations of HSPCs are also expected to be useful for expanding populations of leukemic cells.

Accordingly, in one embodiment there is provided a method of producing a culture of leukemic cells, the method comprising inhibiting the activity and/or expression of cytochrome P450 1B1 (CYP1B1) in a population of one or more leukemic cells. In one embodiment, the method comprises culturing the cells in the presence of a CYP1B1 inhibitor. Optionally, the leukemic cells are acute myelogenous leukemia (AML) cells.

In a further embodiment there is provided a method of producing a culture of leukemic cells, the method comprising increasing the activity and/or expression of Musashi-2 (MSI2) in a population of one or more leukemic cells. In one embodiment, the method comprises culturing the cells in the presence of a MSI2 activator. Optionally, the leukemic cells are acute myelogenous leukemia (AML) cells.

In one embodiment, the culture of leukemic cells may be used in a screening assay for identifying agents that inhibit the proliferation of leukemic cells that may be useful as chemotherapeutic agents. In one embodiment, the method comprises contacting the culture of leukemic cells with a test agent and determining whether the test agent reduces the growth or proliferation of leukemic cells in the cell culture relative to a control.

As used herein, “reducing the growth or proliferation of leukemic cells” refers to a reduction in the number of cells that arise from a leukemic cell as a result of cell growth or cell division and includes cell death. The term “cell death” as used herein includes all forms of killing a cell including necrosis and apoptosis.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

### Example 1: Inhibition of Cytochrome P450 1B1 Oxidase Causes Expansion of Hematopoietic Stem and Progenitor Cells

### Materials and Methods

### Mice

NOD-scid-IL2Rγc^{-/-} (Jackson Laboratory) mice were bred and maintained in the Stem Cell Unit animal barrier facility at McMaster University. All procedures received the approval of the Animal Research Ethics Board at McMaster University.

### Isolation of Primitive Human Hematopoietic Cells and Flow Cytometry

All patient samples were obtained with informed consent and with the approval of local human subject research ethics boards at McMaster University. Human umbilical cord blood mononuclear cells were collected by centrifugation with Ficoll-Paque Plus (GE), followed red blood cell lysis with ammonium chloride (Stemcell Technologies). Cells were then incubated with a cocktail of lineage specific antibodies (CD2, CD3, CD11 b, CD11c, CD14, CD16, CD19, CD24, CD56, CD61, CD66b, and GlyA, Stemcell Technologies) for negative selection of Lin⁻ cells using an EasySep immunomagnetic column (Stemcell Technologies). Live cells were discriminated on the basis of cell size, granularity and, as needed, absence of viability dye 7-AAD (BD Biosciences) uptake. All flow cytometry analysis was performed using a BD LSR II instrument (BD Biosciences). Data acquisition was conducted using BD FACSDiva software (BD Biosciences) and analysis was performed using FlowJo software (Tree Star).

HSPC sorting and qRT-PCR analysis

To quantify MSI2 expression in human HSPCs, Lin⁻ CB cells were stained with the appropriate antibody combinations to resolve HSC (CD34⁺CD38⁻CD45RA⁻ CD90⁺), MPP (CD34⁺CD38⁻CD45RA⁻ CD90⁻), CMP (CD34⁺CD38⁺CD71⁻) and EP (CD34⁺CD38⁺CD71⁺) fractions as similarly described previously (Doulatov et al., 2009; Majeti et al. 2007) with all antibodies from BD Biosciences: CD45RA (HI100), CD90 (5E10), CD34 (581), CD38 (HB7) and CD71 (M-A712). Cell viability was assessed using the viability dye 7AAD (BD Biosciences). All cell subsets were isolated using a BD FACSAria II cell sorter (BD Biosciences) or a MoFlo XDP cell sorter (Beckman Coulter). HemaExplorer (Bagger et al., 2013) analysis was used to confirm MSI2 expression in human HSPCs and across the hierarchy. For all qRT-PCR determinations total cellular RNA was isolated with TRIzol LS reagent according to the manufacturer's instructions (Invitrogen) and cDNA was synthesized using the qScript cDNA Synthesis Kit (Quanta Biosciences). qRT-PCR was done in triplicate with PerfeCTa qPCR SuperMix Low ROX (Quanta Biosciences) with gene specific probes (Universal Probe Library, UPL, Roche) and primers: MSI2 UPL-26, F-ggcagcaagaggatcagg (SEQ ID NO: 1), R-ccgtagagatcggcgaca (SEQ ID NO: 2); HSP90 UPL-46, F-gggcaacacctctacaagga (SEQ ID NO: 3), R-cttgggtctgggtttcctc (SEQ ID NO: 4); CYP1B1 UPL-20, F-acgtaccggccactatcact (SEQ ID NO: 5), R-ctcgagtctgcacatcagga (SEQ ID NO: 6); GAPDH UPL-60, F-agccacatcgctcagacac (SEQ ID NO: 7), R-gcccaatacgaccaaatcc (SEQ ID NO: 8); ACTB (UPL Set Reference Gene Assays, Roche). The mRNA content of samples compared by qRT-PCR was normalized based on the amplification of GAPDH or ACTB.

### Lentivirus Production and Western Blot Validation

MSI2 shRNAs were designed with the Dharmacon algorithm (http://www.dharmacon.com). Predicted sequences were synthesized as complimentary oligonucleotides, annealed and cloned downstream of the H1 promoter of the modfied cppt-PGK-EGFP-IRES-PAC-WPRE lentiviral expression vector (Doulatov et al., 2009). Sequences for the MSI2 targeting and control RFP targeting shRNAs were as follows: shMSI2, 5′-gagagatcccactacgaaa-3′ (SEQ ID NO: 9); shRFP, 5′-gtgggagcgcgtgatgaac-3′ (SEQ ID NO: 10). Human MSI2 cDNA (B0001526; Open Biosystems) was subcloned into the MA bi-directional lentiviral expression vector (Amendola et al., 2005). Human CYP1B1 cDNA (B0012049; Open Biosystems) was cloned in to psMALB (van Galen et al., 2014). All lentivirus was prepared by transient transfection of 293FT (Invitrogen) cells with pMD2.G and psPAX2 packaging plasmids (Addgene) to create VSV-G pseudotyped lentiviral particles. All viral preparations were titrated on HeLa cells before use on cord blood. Standard SDS-PAGE and western blotting procedures were performed to validate the effect of knockdown on transduced NB4 cells (DSMZ) and over expression on 293FT cells. Immunoblotting was performed with anti-MSI2 rabbit monoclonal IgG (EP1305Y, Epitomics) and β-actin mouse monoclonal IgG (ACTBD11B7, Santa Cruz Biotechnology) antibodies. Secondary antibodies used were IRDye 680 goat anti-rabbit IgG and IRDye 800 goat anti-mouse IgG (LI-COR). 293FT and NB4 cell lines tested negative for mycoplasma. NB4 cells were authenticated by ATRA treatment prior to use.

### Cord Blood Transduction

CB transductions were conducted as described previously (Doulatov et al., 2009; Lechman et al., 2012). Briefly, thawed Lin⁻ CB, flow-sorted Lin⁻ CD34⁺CD38⁻or Lin⁻ CD34⁺CD38⁺ cells were prestimulated for 8-12 hours in StemSpan™ medium (StemCell Technologies) supplemented with growth factors Interleukin 6 (IL-6; 20 ng/ml, Peprotech), Stem cell factor (SCF; 100 ng/ml, R&D Systems), Flt3 ligand (FLT3-L; 100 ng/ml, R&D Systems) and Thrombopoietin (TPO; 20 ng/ml, Peprotech). Lentivirus was then added in the same medium at a multiplicity of infection of 30-100 for 24 hours. Cells were then given 2 days post transduction before use in in vitro or in vivo assays. For in vitro CB studies biological (experimental) replicates were performed with three independent CB samples.

### Clonogenic Progenitor Assays

Human clonogenic progenitor cell assays were done in semi-solid methylcellulose medium (Methocult H4434; Stem Cell Technologies) with flow-sorted GFP⁺ cells post transduction (500 cells/ml) or from day seven cultured transduced cells (12000 cells/ml). Colony counts were carried out after 14 days of incubation. CFU-GEMMs can seed secondary colonies due to their limited self-renewal potential (Carow et al., 2993). MSI2 OE and control CFU-GEMM replating for secondary CFU analysis was performed by picking single CFU-GEMMs at day 14 and disassociating colonies by vortexing. Cells were spun and resuspended in fresh methocult, mixed with a blunt end needle and syringe, and then plated into single wells of a 24-well plate. Secondary CFU analysis for shMSI2 and shControl expressing cells was performed by harvesting total colony growth from a single dish (since equivalent numbers of CFU-GEMMs), resuspending cells in fresh methocult by mixing vigorously with a blunt end needle and syringe and then plating into replicate 35 mm tissue culture dishes. In both protocols, secondary colony counts were done following incubation for 10 days. For primary and secondary colony forming assays performed with the AHR agonist 6-Formylindolo(3,2-b)carbazole (FICZ; Santa Cruz Biotechnology), 200 nM FICZ or 0.1% DMSO was added directly to H4434 methocult medium. 2-way ANOVA analysis was performed examining secondary CFU output and FICZ treatment for MSI2 OE or control conditions. Colonies were imaged with a Q-Colour3 digital camera (Olympus) mounted to an Olympus IX5 microscope with a 10× objective lens. Image-Pro Plus imaging software (Media Cybernetics) was used to acquire pictures and subsequent image processing was performed with ImageJ software (NIH).

Lin⁻ CB and Lin⁻ CD34⁺ Suspension Cultures

Transduced human Lin⁻ CB cells were sorted for GFP expression and seeded at a density of 1×10⁵ cells/ml in IMDM 10% FBS supplemented with human growth factors IL-6 (10 ng/ml), SCF (50 ng/ml), FLT3-L (50 ng/ml), and TPO (20 ng/ml) as previously described (Milyaysky et al., 2010). To generate growth curves, every seven days cells were counted, washed, and resuspended in fresh medium with growth factors at a density of 1×10⁵ cells/ml. Cells from suspension cultures were also used in clonogenic progenitor assays, cell cycle and apoptosis assays. Experiments performed on transduced Lin⁻ CD34⁺CB cells used serum free conditions as described in the CB transduction subsection of the Methods. For in vitro CB studies biological (experimental) replicates were performed with three independent CB samples.

### Cell Cycle and Apoptosis Assays

Monitoring cell cycle progression was performed with the addition of BrdU to day 10 suspension cultures at a final concentration of 10 μM. After three hours of incubation, cells were assayed with the BrdU Flow Kit (BD Biosciences) according to the manufacture's protocol. Cell proliferation and quiescence was measured by Ki67 (BD Bioscience) and Hoechst 33342 (Sigma) on day 4 suspension cultures after fixing and permeabilizing cells with the Cytofix/Cytoperm kit (BD Biosciences). For apoptosis analysis, Annexin V (Invitrogen) and 7-AAD (BD Bioscience) staining on day 7 suspension cultures was performed according to the manufacture's protocol.

### Intracellular Flow Cytometry

Lin⁻ CB cells were initially stained with anti-CD34 PE (581) and anti-CD38 APC (HB7) antibodies (BD Biosciences) then fixed with the Cytofix/Cytoperm kit (BD Biosciences) according to the manufacturer's instructions. Fixed and permeabilized cells were immunostained with anti-MSI2 rabbit monoclonal IgG antibody (EP1305Y, Abcam) and detected by Alexa-488 goat anti-rabbit IgG antibody (Invitrogen).

### RNA-Sequencing Data Processing

CD34⁺ cells were transduced with MSI2 OE or KD lentivirus along with their corresponding controls and then sorted for GFP expression 3 days later. Transductions for MSI2 OE and KD were each performed on two independent CB samples. Total RNA from transduced cells (>1×10⁵) was isolated using TRIzol LS as recommended by the manufacturer (Invitrogen), and then further purified using RNeasy columns (Qiagen). Sample quality was assessed using Bioanalyzer RNA Nano chips (Agilent). Paired-end, barcoded RNA-seq sequencing libraries were then generated using the TruSeq RNA Sample Prep Kit (v2) (Illumina) following the manufacturer's protocols starting from 1 ug of total RNA. Quality of library generation was then assessed using a Bioanalyzer platform (Agilent) and Illumina MiSeq-QC run was performed or quantified by qPCR using KAPA quantification kit (KAPA Biosystems). Sequencing was performed using an Illumina HiSeq2000 using TruSeq SBS v3 chemistry at the Institute for Research in Immunology and Cancer's Genomics Platform (University of Montreal) with cluster density targeted at 750 k clusters/mm2 and paired-end 2×100 bp read lengths. For each sample, 90-95M reads were produced and mapped to the hg19 (GRCh37) human genome assembly using CASAVA (version 1.8). Read counts generated by CASAVA were processed in EdgeR (edgeR_3.12.0, R 3.2.2) using TMM normalization, paired design, and estimation of differential expression using a generalized linear model (glmFit). The false discovery rate (FDR) was calculated from the output p-values using the Benjamini-Hochberg method. The fold change of logarithm of base 2 of TMM normalized data (log FC) was used to rank the data from top up-regulated to top down-regulated genes and FDR (0.05) was used to define significantly differentially expressed genes. RNA-seq data has been deposited in NCBI's Gene Expression Omnibus and are accessible through GEO Series accession number GSE70685.

GSEA and iRegulon AHR Target Prediction

iRegulon (Janky et al., 2014) was used to retrieve the top 100 AHR predicted targets with a minimal occurrence count threshold of 5. The data were analyzed using GSEA (Subramanian et al., 2005) with ranked data as input with parameters set to 2000 gene-set permutations.

### GSEA and StemRegenin 1 (SR1) Gene-Sets

The Gene Expression Omnibus (GEO) dataset GSE28359 that contains Affymetrix Human Genome U133 Plus 2.0 Array gene expression data of CD34⁺ cells treated with SR1 at 30 nM, 100 nM, 300 nM and 1000 nM was used to obtain lists of genes differentially expressed in the treated samples compared to the control ones (0 nM) (Boitano et al., 2010). Data were background corrected using Robust Multi-Array Average (RMA) and quantile normalized using the expresso( ) function of the affy Bioconductor package (affy_1.38.1, R 3.0.1). Lists of genes were created from the 150 genes top up regulated and top down regulated by the SR1 treated samples at each dose compared to the non-treated samples (0 nM). The data were analyzed using GSEA with ranked data as input with parameters set to 2000 gene-set permutations. Normalized enrichment score (NES) and false discovery rate (FDR) were calculated for each comparison.

### DMAP Population Comparisons

The GEO dataset GSE24759 that contains Affymetrix GeneChip HT-HG_U133A Early Access Array gene expression data of 38 distinct hematopoietic cell states (Novershtern et al., 2011) was compared to the OE and KD data. GSE24759 data were background corrected using Robust Multi-Array Average (RMA), quantile normalized using the expresso( ) function of the affy Bioconductor package (affy_1.38.1, R 3.0.1), batch corrected using the ComBat( ) function of the sva package (sva_3.6.0) and scaled using the standard score. Bar graphs were created by calculating for significantly differentially expressed genes the number of scaled data that were above (>0) or below (<0) the mean for each population. Percentages indicating time the observed value (set of up or downregulated genes) was better represented in that population than random values were calculated from 1000 trials.

### AHR ChIP-Seq Comparison to Downregulated Gene Sets

A unique list of genes closest to AHR bound regions previously identified from TCDD-treated MCF7 ChIP-seq data (Lo et al., 2012) was used to calculate the overlap with genes showing >1.5-fold down regulation with UM171 (35 nM) and SR1 (500 nM) relative to DMSO treated samples³ as well as with genes significantly down regulated in MSI2 OE versus control treated samples (FDR<0.05). Percentage of down regulated genes with AHR bound regions was then graphed for each gene set. P-values were generated with Fisher's exact test between gene lists.

oPOSSUM Analysis for Promoter AHR Binding Sites in Downregulated Gene Sets

AHR transcription factor binding sites in downregulated gene sets were identified with oPOSSUM-3 (Kwon et al. 2012). Genes showing >1.5-fold down regulation with UM171 (35 nM) and SR1 (500 nM) relative to DMSO treated samples (Fares et al., 2014) were used along with significantly downregulated genes (FDR<0.05) with EdgeR analyzed MSI2 OE versus control treated samples. The three gene lists uploaded into oPOSSUM-3 and the AHR:ARNT transcription factor binding site profile was used with the matrix score threshold set at 80% and analyzing the region 1500 bp upstream and 1000 bp downstream of transcription start site. Percentage of downregulated genes with AHR binding sites in the promoter was then graphed for each gene set. Fisher's exact test was used to identify significant overrepresentation of AHR binding sites in gene lists relative to background.

### Analysis for Human Chimerism

NSG mice were sublethally irradiated (315 cGy) one day prior to intrafemoral injection with transduced cells carried in IMDM 1% FBS at 25 μl per mouse. Injected mice were analyzed for human hematopoietic engraftment at 12-14 weeks post transplantation or 3 and 6.5 weeks for STRC experiments. Mouse bones (femurs, tibiae and pelvis) and spleen were harvested and bones were crushed with a mortar and pestle then filtered in to single cell suspensions. Bone marrow and spleen cells were blocked with mouse Fc block (BD Biosciences) and human IgG (Sigma) and then stained with fluorochrome-conjugated antibodies specific to human hematopoietic cells. For multilineage engraftment analysis, cells from mice were stained with CD45 (H130) (Invitrogen), CD33 (P67.6), CD15 (H198), CD14 (MφP9), CD19 (H1B19), CD235a/GlyA (GA-R2), CD41a (HIP8) and CD34 (581) (BD Biosciences).

### HSC and STRC Xenotransplantations

For MSI2 KD in HSCs 5.0×10⁴ and 2.5×10⁴ sorted Lin⁻ CD34⁺CD38⁻ cells were used per short-hairpin transduction experiment leading to transplantation of day zero equivalent cell doses of 10×10³ and 6.25×10³, respectively per mouse. For STRC LDA transplantation experiments 1×10⁵ sorted CD34⁺CD38⁺ cells were used per control and MSI2 OE transduction. After assessing levels of gene transfer, day zero equivalent GFP⁺ cell doses were calculated to perform the LDA. Recipients with greater than 0.1% GFP⁺CD45^{+/-} cells were considered to be repopulated. For STRC experiments that readout extended engraftment at 6.5 weeks, 2×10⁵ CD34⁺CD38⁺ cells were used per OE and control transduction allowing for non-limiting 5×10⁴ day zero equivalent cell doses per mouse. For HSC expansion and LDA experiments CD34⁺CD38⁻ cells were sorted and transduced with MSI2 OE or control vectors (50,000 cells per condition) for three days and then analyzed for gene-transfer levels (% GFP^{+/-}) and primitive cell marker expression (% CD34 and CD133). To ensure equal numbers of GFP⁺ cells were transplanted in both control and MSI2 OE mice, we added identically cultured GFP⁻ cells to the MSI2 culture to match % GFP⁺of the control culture (necessary due to the differing efficiency of transduction). The adjusted OE culture was recounted and aliquoted (63,000 cells) to match the output of half the control culture. Three day 0 equivalent GFP⁺ cell doses (1000, 300 and 62) were then transplanted per mouse to perform the D3 primary LDA. A second aliquot of the adjusted OE culture was then taken and put in to culture in parallel with the remaining half of the control culture for performing another LDA after 7 days of growth (10 days total growth, D10 primary LDA). Altogether, 4 cell doses were transplanted, if converted back to day 0 equivalents these equaled approximately 1000, 250, 100, and 20 GFP⁺ cells per mouse, respectively. Pooled bone marrow from six engrafted primary mice that received D10 cultured control or MSI2 OE cells (from 2 highest doses transplanted) was aliquoted in to 4 cell doses of 15 million, 10 million, 6 million, 2 million and 1 million cells. Numbers of GFP⁺ cells within primary mice was estimated from nucleated cell counts obtained from NSG femurs, tibias and pelvis and from Colvin et al., 2004. The cutoff for HSC engraftment was a demonstration of multilineage reconstitution which was set at BM having >0.1% GFP⁺ CD33⁺ and >0.1% GFP⁺CD19⁺ cells. Assessment of HSC and STRC frequency was carried out by using ELDA software (Hu & Smyth, 2009). For all mouse transplantation experiments, mice were aged (6-12 wk) and sex matched. All transplanted mice were included for analysis unless mice died from radiation sickness before the experimental endpoint. No randomization or blinding was performed for animal experiments. Approximately 3-6 mice were used per cell dose for each CB transduction and transplantation experiment.

### UV Cross-Linking Immunoprecipitation Sequencing (CLIP-Seq) Library Preparation

CLIP-seq was performed as previously described (Yeo et al., 2009). Briefly, 25 million NB4 cells, a transformed human cell line of hematopoietic origin, were washed in PBS and UV-cross-linked at 400mJ/cm² on ice. Cells were pelleted, lysed in wash buffer (PBS, 0.1% SDS, 0.5% Na-Deoxycholate, 0.5% NP-40), DNAse treated, and supernatants from lysates were collected for immunoprecipitation. MSI2 was immunoprecipitated overnight using 5 μg of anti-MSI2 antibody (EP1305Y, Abcam) and Protein A Dynabeads (Invitrogen). Beads containing immunoprecipated RNA were washed twice with wash buffer, high-salt wash buffer (5×PBS, 0.1% SDS, 0.5% Na-Deoxycholate, 0.5% NP-40), and PNK buffer (50 mM Tris-CI pH 7.4, 10 mM MgCl₂, 0.5% NP-40). Samples were then treated with 0.2 U MNase for 5 minutes at 37 degrees with shaking to trim immunopreciptated RNA. MNase inactivation was then carried out with PNK+EGTA buffer (50 mM Tris-CI pH 7.4, 20 mM EGTA, 0.5% NP-40). The sample was dephosphorylated using alkaline phosphatase (CIP, NEB) at 37 degrees for 10 followed by washing with PNK+EGTA, PNK buffer, and then 0.1 mg/mL BSA in nuclease free water. 3′ RNA linker ligation was performed at 16 degrees overnight with the following adapter: 5′ P-UGGAAUUCUCGGGUGCCAAGG-puromycin (SEQ ID NO: 11). Samples were then washed with PNK buffer, radiolabelled using P32-y-ATP (Perkin Elmer), run on a 4-12% Bis-Tris gel and then transferred to a nitrocellulose membrane. The nitrocellulose membrane was developed via autoradiography and RNA-protein complexes 15-20 kDa above the molecular weight of MSI2 was extracted with Proteinase K followed by RNA extraction with acid phenol-chloroform. A 5′ RNA linker (5′ HO-GUUCAGAGUUCUACAGUCCGACGAUC-OH) (SEQ ID NO: 12) was ligated to the extracted RNA using T4 RNA ligase (Fermentas) for two hours and the RNA was again purified using acid phenol-chloroform. Adapter ligated RNA was re-suspended in nuclease free water and reverse transcribed using Superscript III reverse transcriptase (Invitrogen). 20 cycles of PCR were performed using NEB Phusion Polymerase using a 3′ PCR primer that contained a unique Illumina barcode sequence. PCR products were run on an 8% TBE gel. Products ranging between 150-200 bp were extracted using the QIAquick gel extraction kit (Qiagen) and re-suspended in nuclease free water. Two separate libraries were prepared and sent for single-end 50 bp Illumina sequencing at the Institute for Genomic Medicine at the University of California, San Diego. 47,098,127 reads from the first library passed quality filtering of which 73.83% mapped uniquely to the human genome. 57,970,220 reads from the second library passed quality filtering of which 69.53% mapped uniquely to the human genome. CLIP-data reproducibility was verified through high correlation between gene RPKMs and statistically significant overlaps in the clusters and genes within replicates. CLIP-seq data has been deposited in NCBI's Gene Expression Omnibus and are accessible through GEO Series accession number GSE69583.

### CLIP-Seq Mapping and Cluster Identification

Before sequence alignment of CLIP-seq reads to the human genome was performed, sequencing reads from libraries were trimmed of polyA tails, adapters, and low quality ends using Cutadapt with parameters—match-read-wildcards—times 2-e 0-O 5—quality-cutoff’ 6-m 18-b TCGTATGCCGTCTTCTGCTTG-b ATCTCGTATGCCGTCTTCTGCTTG-b CGACAGGTTCAGAGTTCTACAGTCCGACGATC-b TGGAATTCTCGGGTGCCAAGG-b AAAAAAAAAAAAAAAAAAAAAAAAAAAAAAAAAAAAAAAAAAAAAAAAAA-b TTTTTTTTTTTTTTTTTTTTTTTTTTTTTTTTTTTTTTTTTTTTTTTTTT. (SEQ ID NOs: 13-18). Reads were then mapped against a database of repetitive elements derived from RepBase (version 18.05). Bowtie (version 1.0.0) with parameters-S-q-p 16-e 100-l 20 was used to align reads against an index generated from Repbase sequences (Langmead et al., 2001). Reads not mapped to Repbase sequences were aligned to the hg19 human genome (UCSC assembly) using STAR (version 2.3.0e) (Dobin et al., 2013) with parameters—outSAMunmapped Within—outFilterMultimapNmax 1—outFilterMultimapScoreRange 1. To identify clusters in the genome of significantly enriched CLIP-seq reads, reads that were PCR replicates were removed from each CLIP-seq library using a custom script of the same method as³³, otherwise reads were kept at each nucleotide position when more than one read's 5′ end was mapped. Clusters were then assigned using the CLIPper software with parameters—bonferroni—superlocal—threshold-(Lovci et al., 2013). The ranked list of significant targets was calculated assuming a Poisson distribution, where the observed value is the number of reads in the cluster, and the background is the number of reads across the entire transcript and or across a window of 1000 bp+/− the predicted cluster.

### Gene Annotations for CLIP-Seq

Transcriptomic regions and gene classes were defined using annotations found in gencode v17. Depending on the analysis clusters were either associated by the Gencode annotated 5′ UTR, 3′ UTR, CDS or intronic regions. If a cluster overlapped multiple regions, or a single part of a transcript was annotated as multiple regions, clusters were iteratively assigned first as CDS, then 3′ UTR, 5′ UTR and finally as proximal (<500 bases from an exon) and distal (>500 bases from an exon) introns. Overlapping peaks were calculated using bedtools and pybedtools (Quinlan et al., 2010; Dale et al., 2011).

### Gene Ontology Analysis for CLIP-Seq

Significantly enriched gene ontology (GO) terms were identified using a hypergeometric test that compared the number of genes that were MSI2 targets in each GO term to genes expressed in each GO term as the proper background. Expressed genes were identified by using the control samples in SRA study SRP012062. Mapping was performed identically to CLIP-seq mapping, without peak calling and changing the STAR parameter outFilterMultimapNmax to 10. Counts were calculated with feature Counts (Liao et al., 2014) and RPKMs were then computed. Only genes with a mean RPKM>1 between the two samples were used in the background expressed set.

### De-Novo Motif and Conservation Analysis for CLIP-Seq

Randomly located clusters within the same genic regions as predicted MSI2 clusters were used to calculate a background distribution for motif and conservation analyses. Motif analysis was performed using the HOMER algorithm as in Lovci et al., 2013. For evolutionary sequence conservation analysis the mean (mammalian) phastCons score for each cluster was used.

### Immunofluorescence

CD34⁺ cells (>5×10⁴) were transduced with MSI2 OE or control lentivirus, after 3 days GFP⁺ cells were sorted and then put back in to StemSpan medium containing growth factors IL-6 (20 ng/ml), SCF (100 ng/ml), FLT3-L (100 ng/ml) and TPO (20 ng/ml). A minimum of 10,000 cells were used for immunostaining at culture days 3 and 7 post GFP-sort. Cells were fixed in 2% PFA for 10 minutes, washed with PBS and then cytospun on to glass slides. Cytospun cells were then permeabilized (PBS 0.2% Triton X-100) for 20 minutes, blocked (PBS 0.1% saponin 10% donkey serum) for 30 minutes and stained with primary antibodies (anti-CYP1B1 antibody, EPR14972, Abcam; anti-HSP90 antibody, 68/hsp90, BD Biosciences) in PBS 10% donkey serum for 1 hour. Detection with secondary antibody was performed in PBS 10% donkey serum with Alexa-647 donkey anti-rabbit antibody or Alexa-647 donkey anti-mouse antibody for 45 minutes. Slides were mounted with Prolong Gold Antifade containing DAPI (Invitrogen). Several images (200-1000 cells total) were captured per slide at 20× magnification using an Operetta HCS Reader (Perkin Elmer) with epifluorescence illumination and standard filter sets. Columbus software (Perkin Elmer) was used to automate the identification of nuclei and cytoplasm boundaries in order quantify mean cell fluorescence.

### Luciferase Reporter Gene Assay

A 271 bp region of the CYP1B1 3′UTR that flanked CLIP-seq identified MSI2 blinding sites was cloned from human HEK293FT genomic DNA using the forward primer GTGACACAACTGTGTGATTAAAAGG (SEQ ID NO: 19) and reverse primer TGATTTTTATTATTTTGGT AATGGTG (SEQ ID NO: 20) and placed downstream of renilla luciferase in the dual-luciferase reporter vector, pGL4 (Promega). A 271 bp geneblock (IDT) with 6 TAG>TCC mutations was cloned in to pGL4 using XbaI and NotI. The HSP90 3′UTR was amplified from HEK293FT genomic DNA with the forward primer TCTCTGGCTGAGGGATGACT (SEQ ID NO: 21) and reverse primer TTTTAAGGCCAAGGAATTAAGTGA (SEQ ID NO: 22) and cloned in pGL4. A geneblock of the HSP90 3′UTR (IDT) with 14 TAG>TCC mutations was cloned in to pGL4 using SfaAI and NotI. Co-transfection of wild type or mutant luciferase reporter (40 ng) and control or MSI2 OE lentiviral expression vector (100 ng) was performed in the non-Musashil and 2 expressing NIH-3T3 cell line. 50,000 cells were used per co-transfection. Reporter activity was measured using the Dual-Luciferase Reporter Assay System (Promega) 36-40 hours later.

MSI2 OE Suspension Cultures with AHR Antagonist SR1 and Agonist FICZ

For MSI2 OE cultures with the AHR antagonist SR1, Lin⁻ CD34⁺ cells were transduced with MSI2 OE or control lentivirus in medium supplemented with SR1 (750 nM; Abcam) or DMSO vehicle (0.1%). GFP⁺ cells were isolated (20,000 cells per culture) and allowed to proliferate with or without SR1 for an additional 7 days at which point they were counted and immunophenotyped for CD34 and CD133 expression. For MSI2 OE cultures with the AHR agonist FICZ, Lin⁻ CD34⁺ cells were transduced with MSI2 OE or control lentivirus. GFP⁺ cells were isolated (20,000 cells per culture) and allowed to proliferate with FICZ (200 nM; Santa Cruz Biotechnology) or DMSO (0.1%) for an additional 3 days at which point they were immunophenotyped for CD34 and CD133 expression.

HSPC Expansion with (E)-2,3′,4,5′-Tetramethoxystilbene (TMS)

Lin⁻ CD34⁺ cells were cultured for 72 hours (lentiviral treated but non-transduced flow-sorted GFP⁻ cells) in StemSpan medium containing growth factors IL-6 (20 ng/ml), SCF (100 ng/ml), FLT3-L (100 ng/ml) and TPO (20 ng/ml) before the addition of the CYP1B1 inhibitor TMS (Abcam) at a concentration of 10 μM or mock treatment with 0.1% DMSO. Equal numbers of cells (12,000 per condition) were then allowed to proliferate for 7 days at which point they were counted and immunophenotyped for CD34 and CD133 expression.

### Statistical Analysis

Unless stated otherwise (i.e., analysis of RNA-seq and CLIP-seq data sets), all statistical analysis was performed using GraphPad Prism (GraphPad Software version 5.0). Unpaired student t-tests or Mann-Whitney tests were performed with p<0.05 as the cut-off for statistical significance.

### Results & Discussion

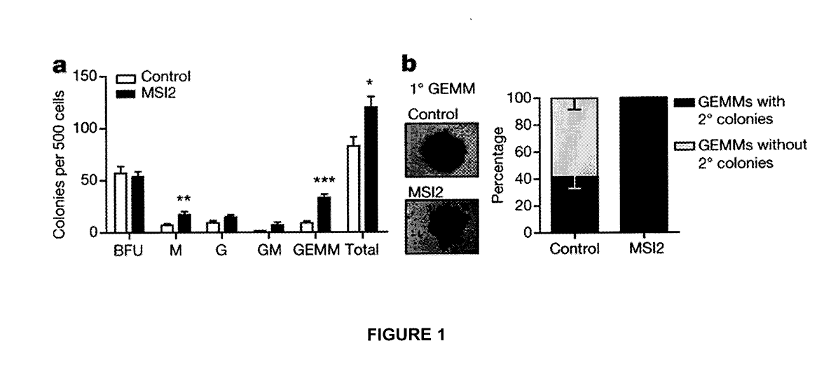

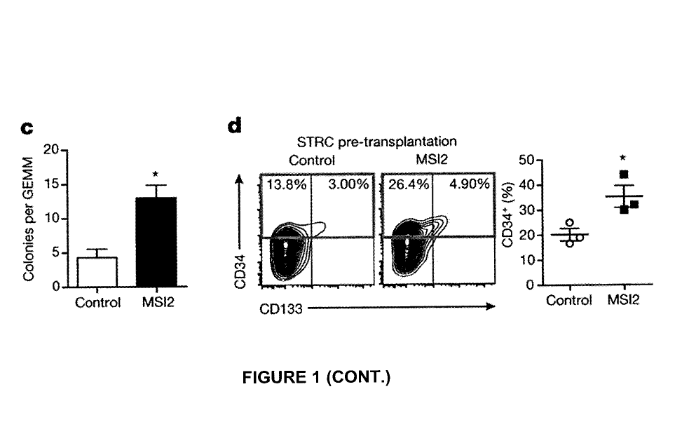

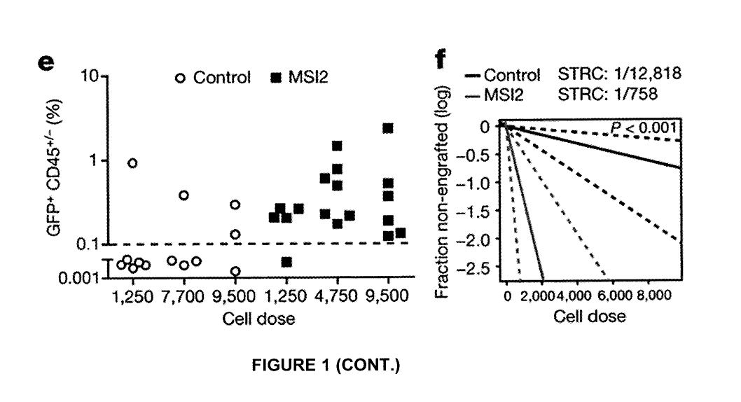

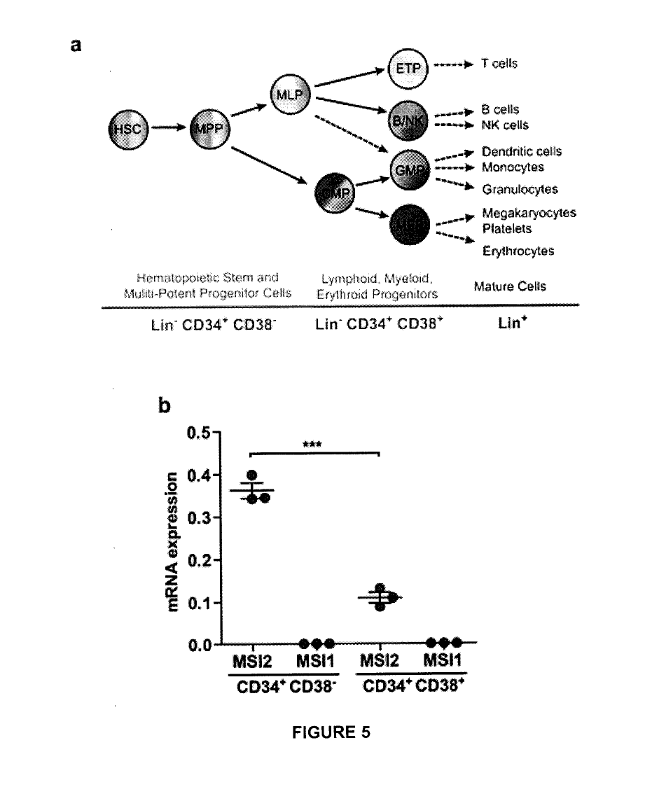

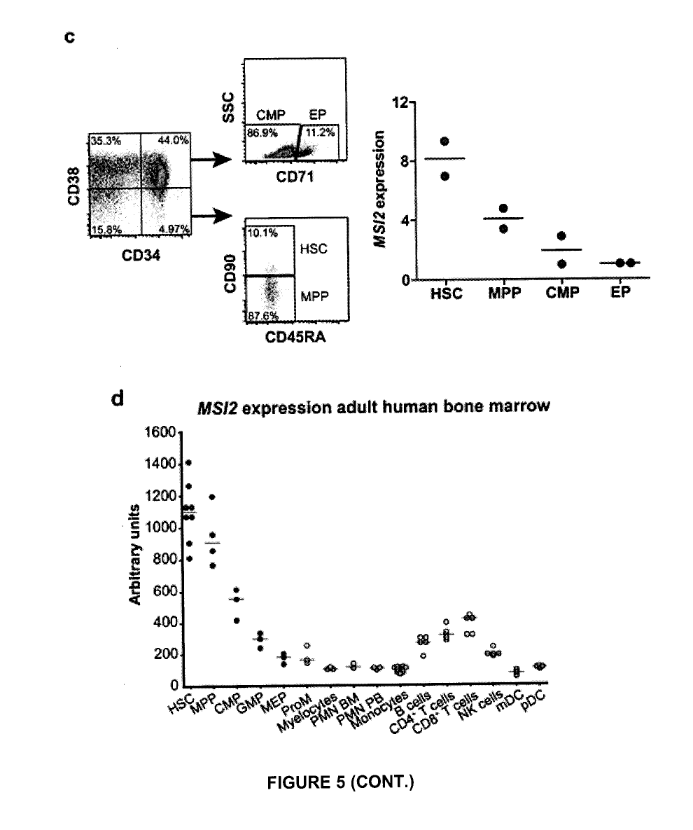

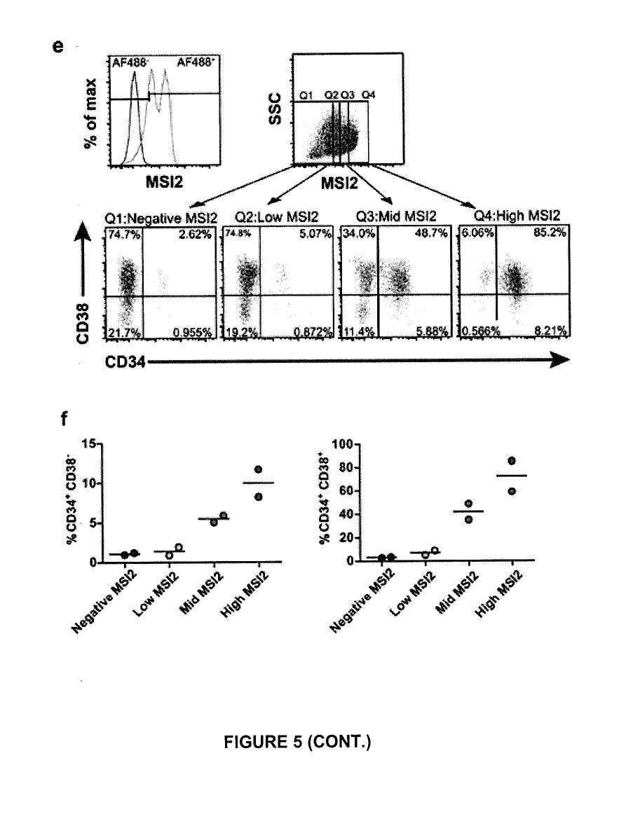

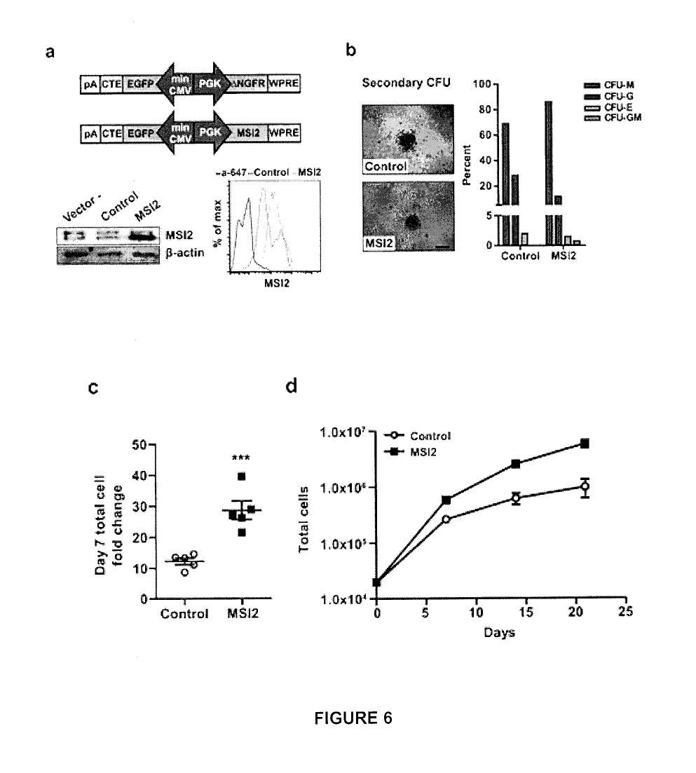

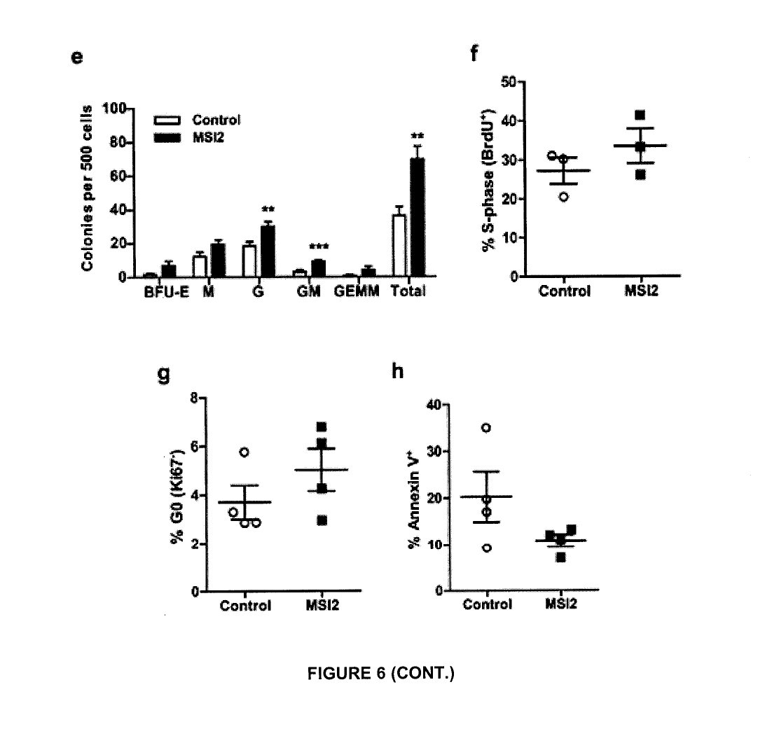

The role of MSI2 in post-transcriptionally controlling human HSPC self-renewal was investigated as it is known to regulate mouse HSCs, (Hope et al., 2010, de Andres-Aguayo et al., 2011; Park et al., 2014) and is predicted to impact mRNA translation (Ohyama et al., 2012). MSI2 was present and elevated in primitive CB HSPCs and decreased during differentiation, whereas its paralog, MSI1, was not expressed (FIG. 5a-f). Lentiviral overexpression (OE) of MSI2 resulted in a 1.5-fold increase in colony forming units (CFU) relative to control, principally due to a 3.7-fold increase in the most primitive CFU-Granulocyte Erythrocyte Monocyte Megakaryocyte (GEMM) colony type (FIG. 6a, FIG. 1a). Remarkably, 100% of MSI2 OE CFU-GEMMs generated secondary colonies compared to only 40% of controls. In addition, MSI2 OE yielded 3-fold more colonies per re-seeded CFU-GEMM (FIG. 1b, c, FIG. 6b). During in vitro culture MSI2 OE resulted in 2.3- and 6-fold more cells relative to control at the 7 and 21-day time points, respectively (FIG. 6c, d). Moreover after 7 days in culture MSI2 OE yielded a cumulative 9.3-fold increase in colony forming cells in the absence of changes in cell cycling or death (FIG. 6e-h). Altogether, this data demonstrates that enforced expression of MSI2 has potent self-renewal effects on early progenitors and promotes their in vitro expansion.

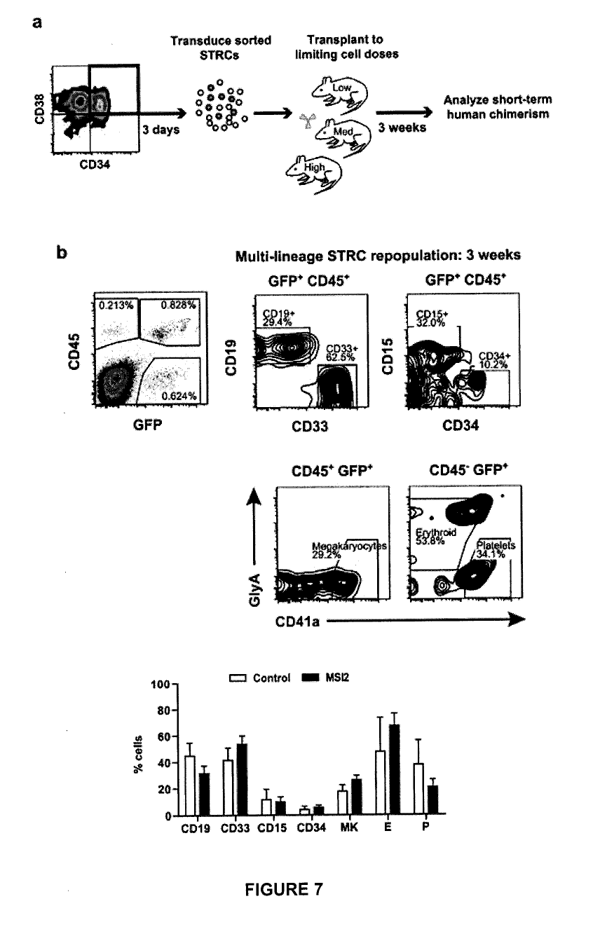

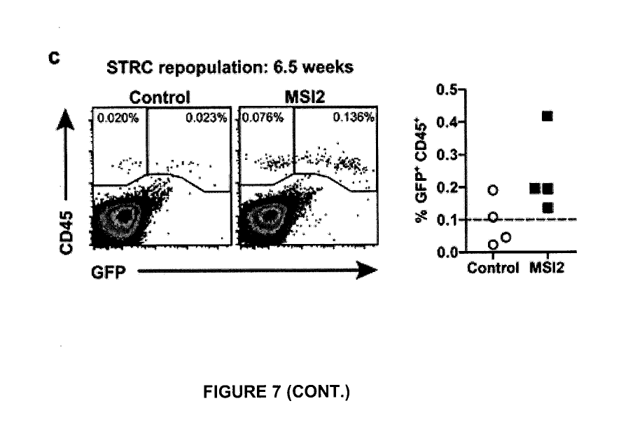

Short-term repopulating cells (STRC) produce a transient multilineage graft in NOD-scid-IL2Rγc^{-/-} (NSG) mice (Glimm et al., 2001), and in patients reconstitute granulocytes and platelets critical for preventing post-transplant infection and bleeding (Miller et al., 2013). STRCs overexpressing MSI2 exhibited 1.8-fold more primitive CD34⁺ cells post-infection and a dramatic 17-fold increase in functional STRCs relative to control as readout through limiting dilution analysis (LDA) of human chimerism at 3 weeks post-transplant (FIG. 1d-f, FIG. 7a, b). Furthermore, at a protracted engraftment readout time of 6.5 weeks at non-limiting transplant doses, 100% of MSI2 OE STRC transplanted mice were engrafted compared to only 50% of controls, indicating MSI2 OE extended the duration of STRC-mediated engraftment (FIG. 7c).

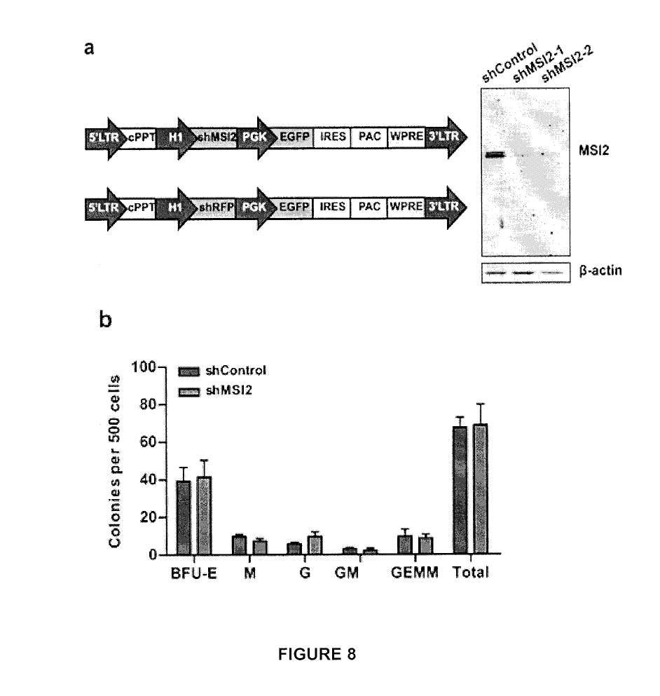

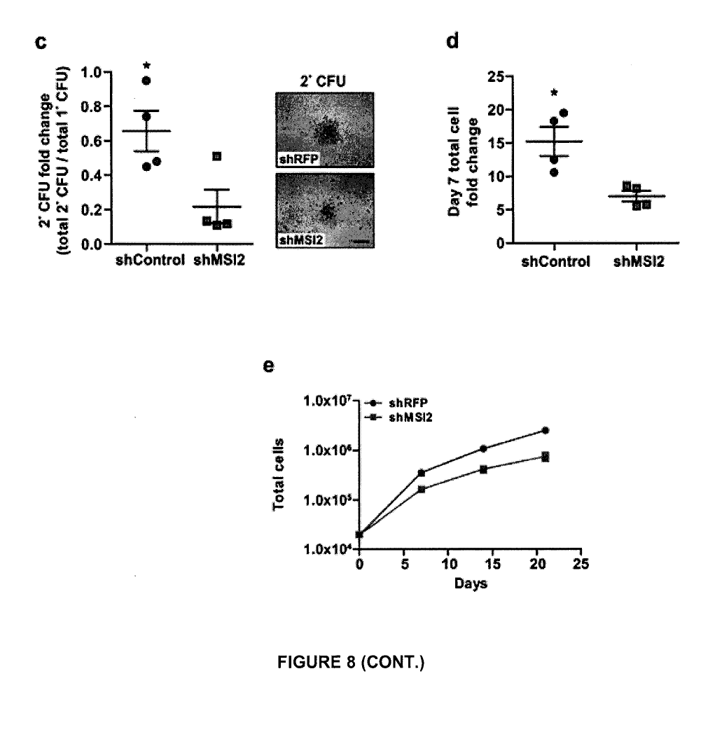

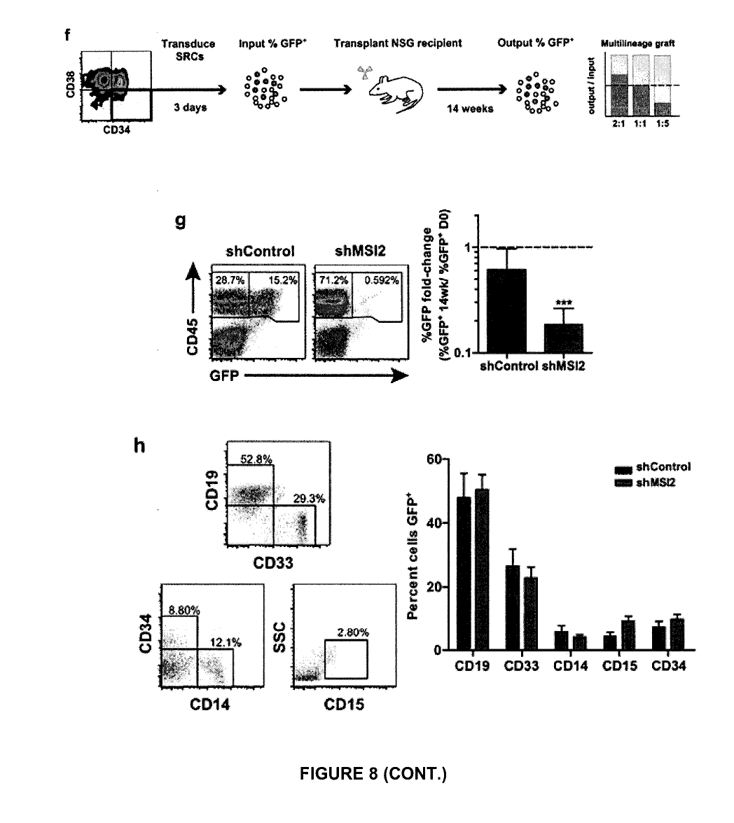

Next, the effect of shRNA-induced MSI2 knockdown (KD) on HSPC function was investigated. MSI2 OE did not alter clonogenic potential but did decrease CFU replating 3-fold (FIG. 8a-c). When effects on more primitive culture-initiating cells were explored we found MSI2 KD significantly decreased cell number over culture (FIG. 8d, e) independent of increased death or altered cell cycling. Upon transplantation, engrafted MSI2 KD GFP⁺ cells showed no evidence of lineage skewing, yet were strikingly reduced relative to the percentage of GFP⁺ cells initially transplanted (FIG. 8f-h). Combined, the in vitro and in vivo data show that MSI2 KD reduces self-renewal in early progenitors and HSCs.

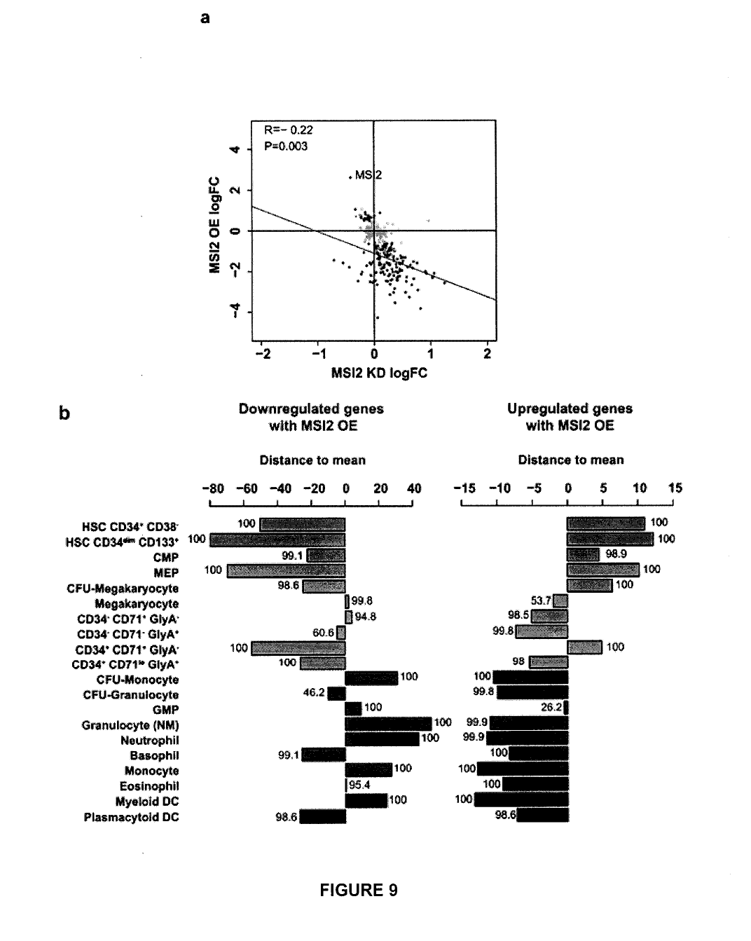

To characterize the earliest transcriptional changes induced by modulating MSI2 expression, RNA-seq was performed on CD34⁺ MSI2 OE and KD cells immediately post-transduction. MSI2 OE-induced transcriptional changes anti-correlated with those of MSI2 KD, suggesting OE and KD have opposite effects (FIG. 9a). When compared to transcriptome data from 38 human hematopoietic cell subpopulations (Novershtern et al., 2011), it was observed that genes significantly upregulated upon MSI2 OE and downregulated upon MSI2 KD were exclusively enriched for those highly expressed in HSC and other primitive CD34⁺ populations (FIG. 9b).

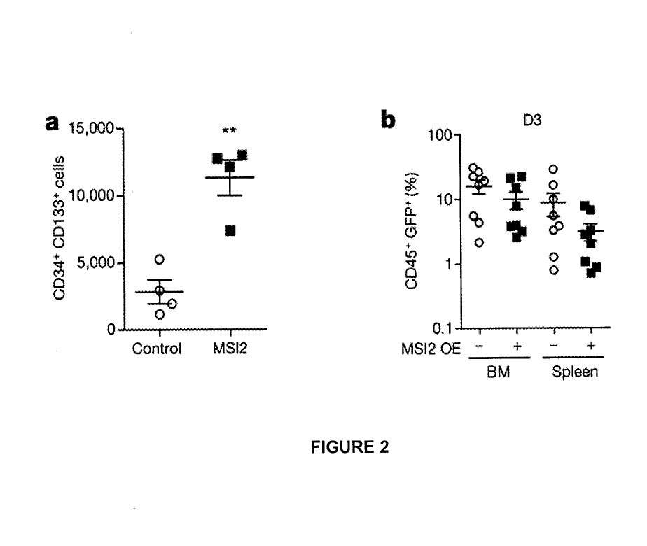

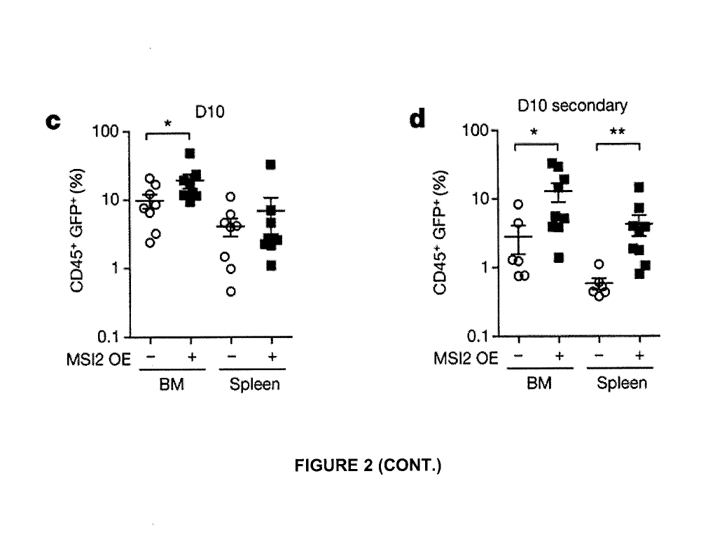

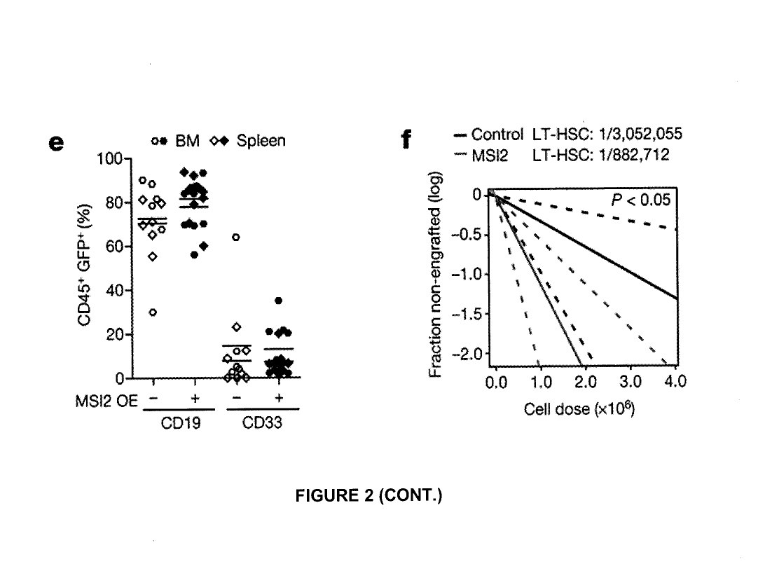

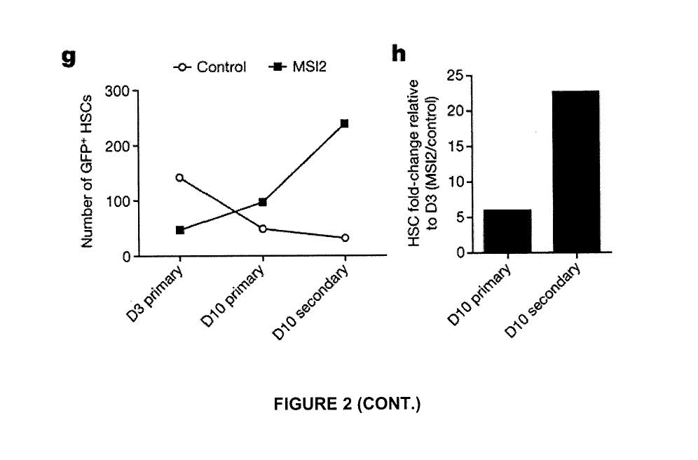

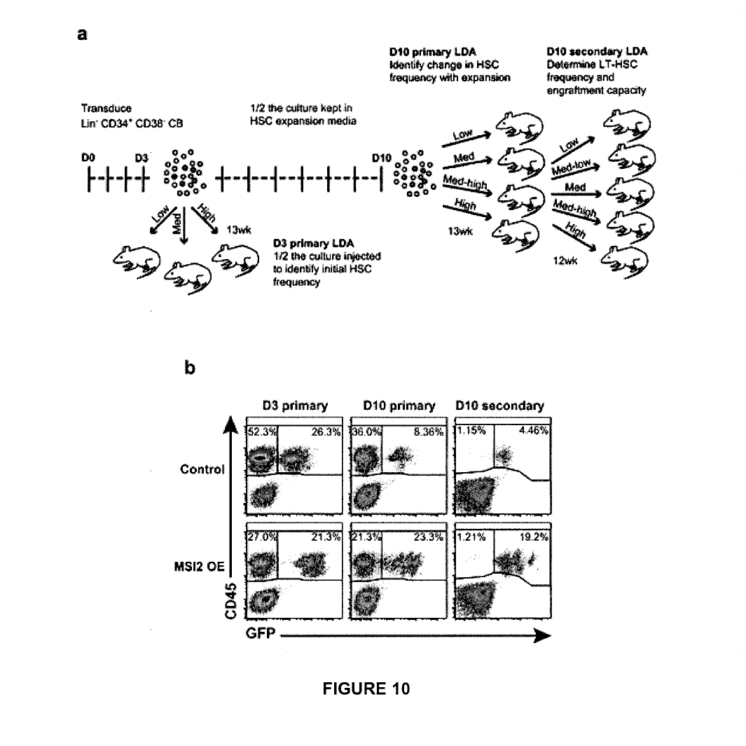

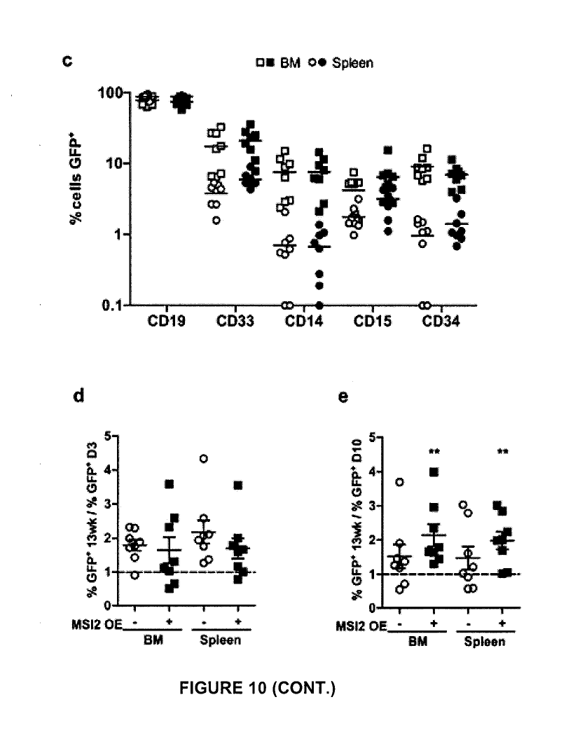

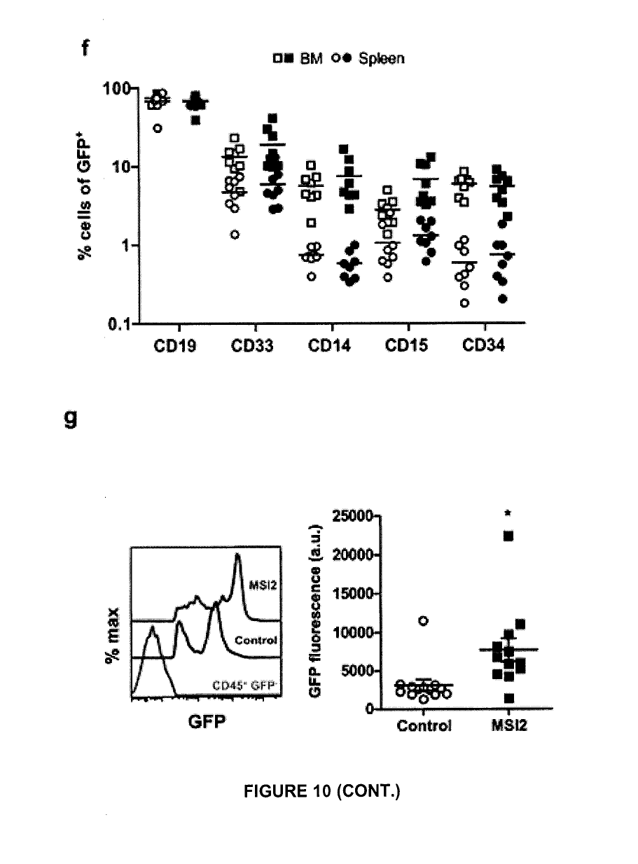

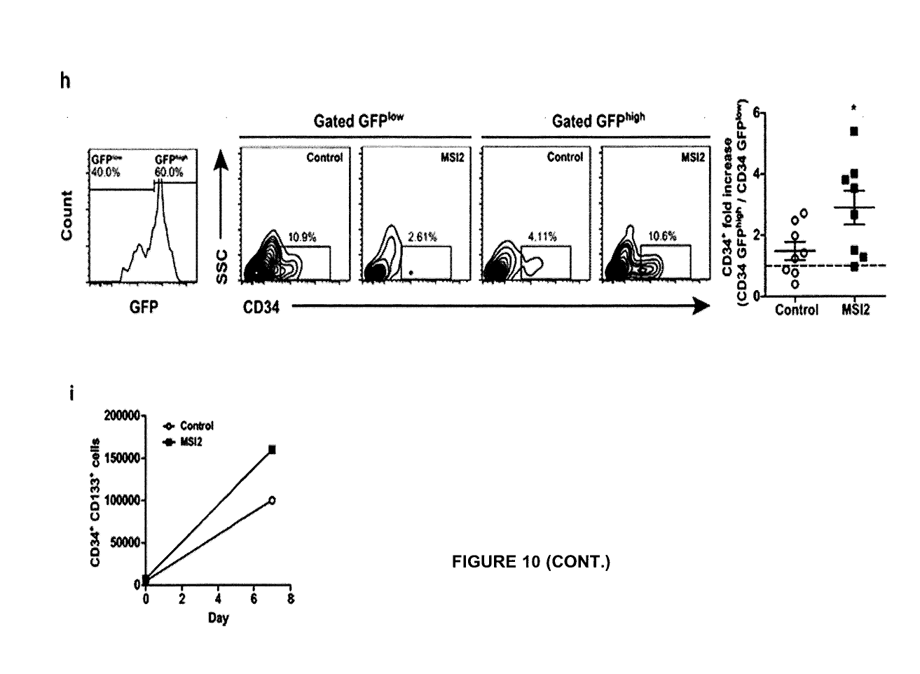

Since MSI2 OE conferred an HSC gene expression program, it was hypothesized that it could facilitate HSC expansion ex vivo. Remarkably, MSI2 OE induced a 4-fold increase in CD34⁺CD133⁺ phenotypic HSCs relative to control after 7 days of culture (FIG. 2a). An LDA was performed to define functional HSC frequency before (day 3 post transduction, D3) and after 7 days of ex vivo culture (day 10, D10, FIG. 10a). D3 recipients displayed no altered engraftment as a result of MSI2 OE, however, recipients of MSI2 OE D10 expanded cells displayed multiple phenotypes of enhanced reconstitution relative to control, including a 2-fold increase in BM GFP⁺ levels without changes to lineage output, proportionally more GFP⁺ cells within the human graft relative to pre-transplant D10 levels, greater GFP mean fluorescence intensity and enrichment of CD34 expression in GFP^{high} cells (FIG. 2b, c, FIG. 10b-h). As the lentiviral construct design ensures GFP levels mirror MSI2's, this indicates high levels of MSI2 impart enhanced competitiveness and are conducive to in vivo HSPC activity. Importantly, D10 MSI2 OE cultures contained more CD34⁺CD133⁺ cells prior to transplant (FIG. 10i), and in accordance, the HSC frequency in D10 MSI2 OE cultures was increased 2-fold relative to the D3 culture time point, whereas control cultures displayed a 3-fold decrease. These results demonstrate that MSI2 OE facilitated an ex vivo net 6-fold increase in HSCs relative to control (FIG. 2g, h).

Secondary LDA transplants were performed to fully explore the effects MSI2 OE and culturing has on self-renewal and long-term HSCs (LT-HSC). Robust engraftment with MSI2 OE showed no evidence of altered myelo-lymphopoiesis or leukemic development (FIG. 2e). Secondary LDA measurements revealed BM GFP⁺ percentage increased 4.6-fold and LT-HSC frequency 3.5-fold with MSI2 OE compared to controls (FIG. 2d, e, f). This corresponds to MSI2 OE GFP⁺ HSCs having expanded in primary mice 2.4-fold over input as compared to a decrease of 1.5-fold for control HSCs (FIG. 2g). The level of MSI2 OE-induced in vivo fold expansion remains in line with the behaviour of uncultured HSCs, which undergo similarly controlled expansion during passage in mice (Fares et al., 2014; Cashman et al., 1999; Holyoake et al., 1999). Finally, when accounting for the total change in GFP⁺ HSCs upon ex vivo culture, MSI2 OE provided a cumulative 23-fold-expansion of secondary LT-HSC relative to control (FIG. 2g, h) indicating elevated MSI2 provides a significant self-renewal advantage to functional HSCs during ex vivo culture.

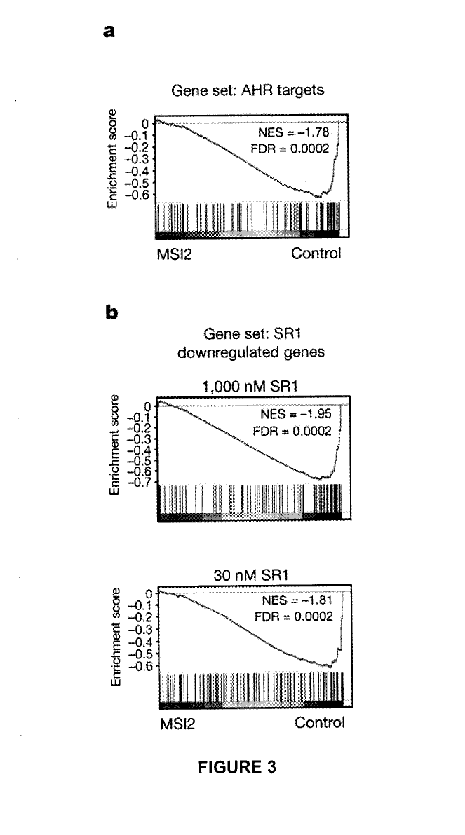

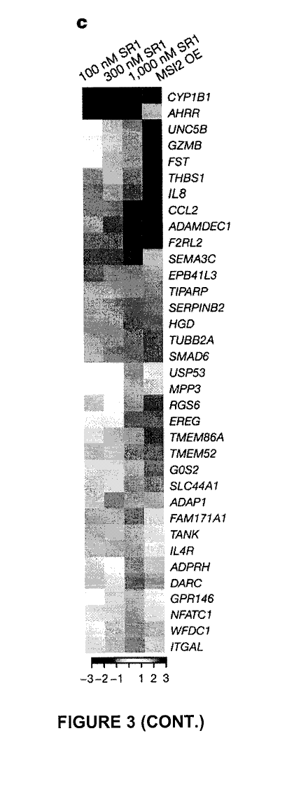

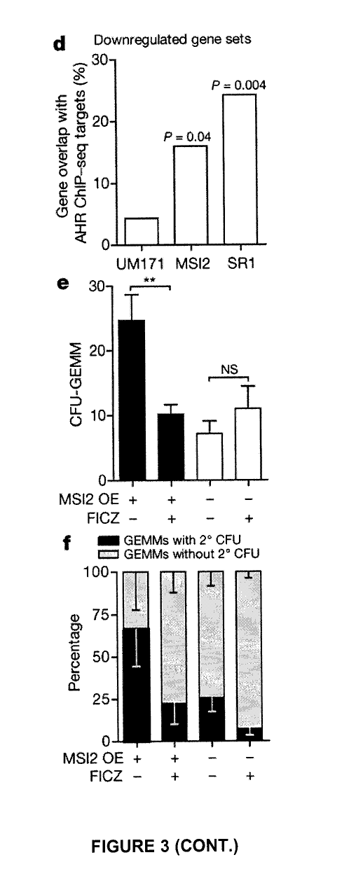

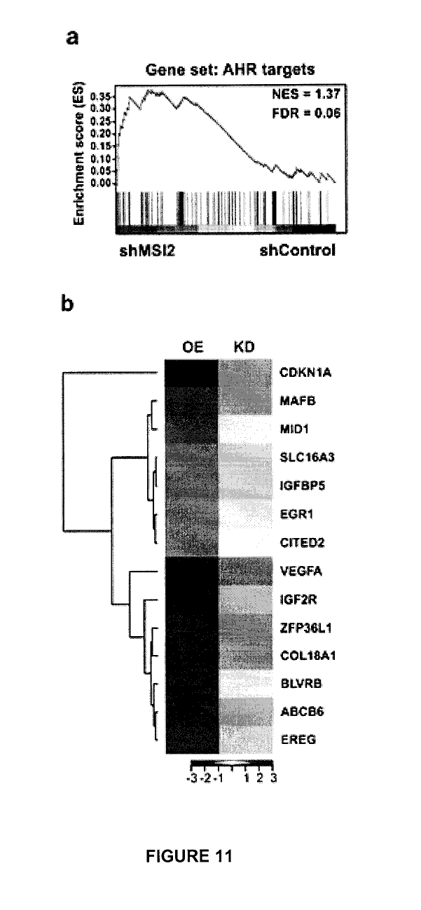

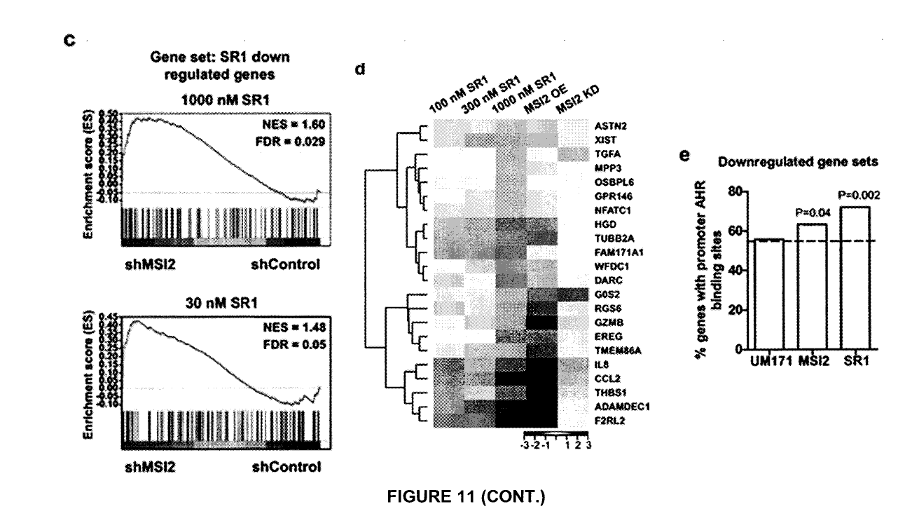

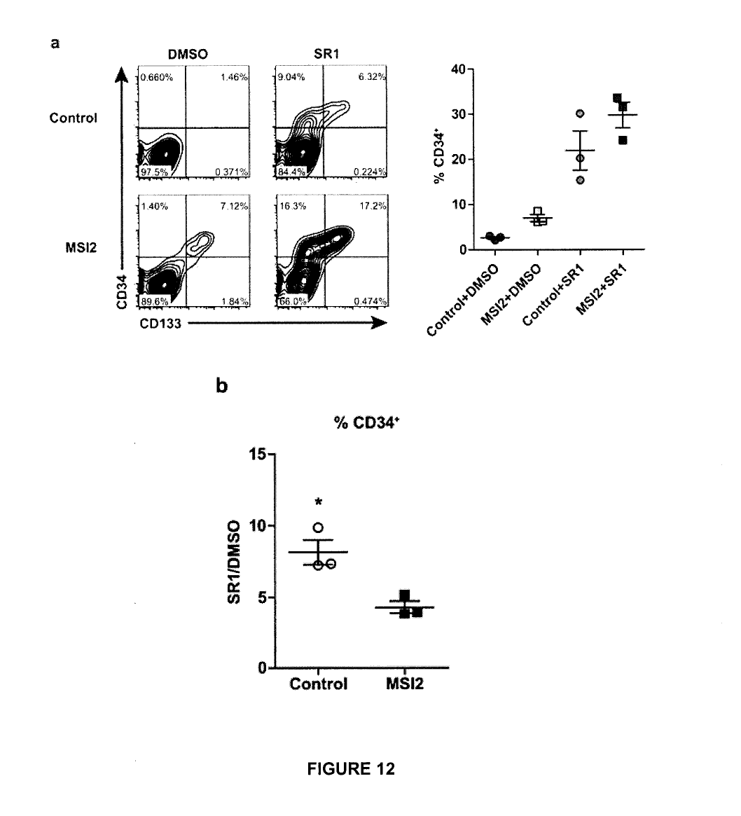

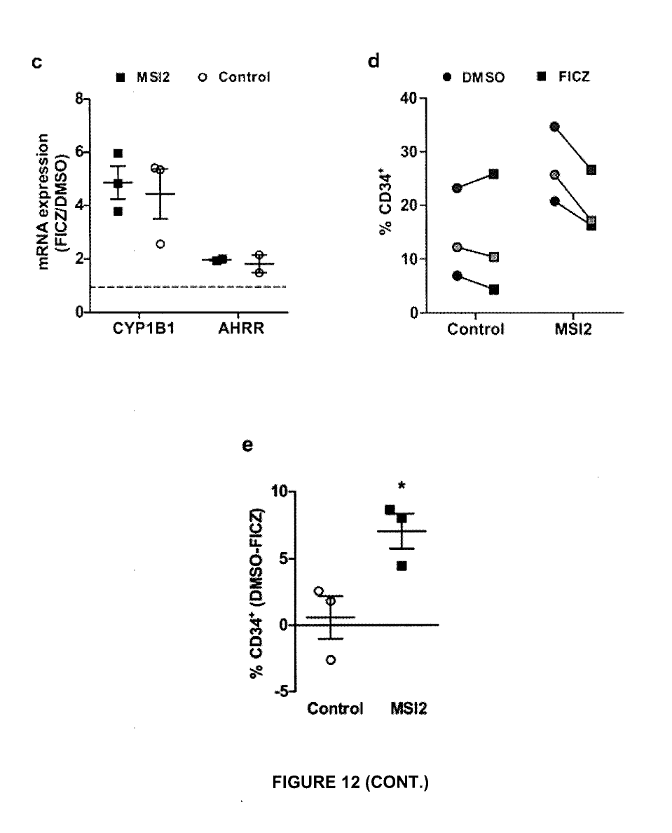

To gain mechanistic insights MSI2 OE-induced differentially expressed genes were examined and found Cytochrome P450 1B1 Oxidase (CYP1B1), an effector of AHR signaling (Mimura et al., 2003) amongst the most repressed. Pathway analysis revealed many predicted AHR targets were enriched in MSI2 OE downregulated (FIG. 3a) and MSI2 KD upregulated gene sets (FIG. 11a, b). Binding of the nuclear receptor transcription factor AHR with StemRegenin 1 (SR1) inhibits AHR target gene activation leading to human HSPC expansion in culture (Boitan et al., 2010). Gene set enrichment analysis (GSEA) revealed MSI2 OE downregulated genes significantly matched the SR1 downregulated gene set in an SR1 dose-dependent manner (FIG. 3b, c), whereas MSI2 KD induced the opposite expression profile (FIG. 11c, d). The overlap of downregulated genes with ChIP-seq-identified AHR targets (Lo et al., 2012) was examined. This comparison was extended to downregulated genes upon treatment with UM171, another compound that expands HSPCs independent of AHR (Fares et al., 2014). Direct transcriptional targets of AHR were enriched in the MSI2 OE and SR1 downregulated gene sets by 3.8 and 5.6-fold, respectively compared to UM171, an overrepresentation maintained for predicted AHR targets and one that suggests MSI2 OE expands HSPCs through AHR signaling attenuation (FIG. 3d, FIG. 11e). Furthermore, SR1 treatment increased the percentage of CD34⁺ cells 8-fold for control cultures compared to only 4-fold with MSI2 OE (FIG. 12a, b), a finding which indicates a redundancy in SR1 and MSI2 OE activity on HSPCs and provides evidence they act on the same pathway.

To further elucidate the AHR connection, MSI2 OE and control cultures were treated with the AHR agonist 6-Formylindolo(3,2-b)carbazole (FICZ), whose induction of canonical AHR targets in MSI2 OE cells, demonstrates they remain competent for AHR activation (FIG. 12c). Interestingly, FICZ induced a dramatic reversal of the MSI2 OE-mediated increases in primary CFU-GEMMs and their replating capacities (FIG. 3e, f). Furthermore, FICZ-treated MSI2 OE cultures displayed greater losses in phenotypic HSPCs compared to controls which showed no change (FIG. 12d, e). Altogether, these results demonstrate agonist-induced restoration of AHR activity reduces MSI2 OE's pro-self-renewal effects and strongly supports downregulation of AHR signaling as the mechanism through which MSI2 OE achieves HSPC expansion.

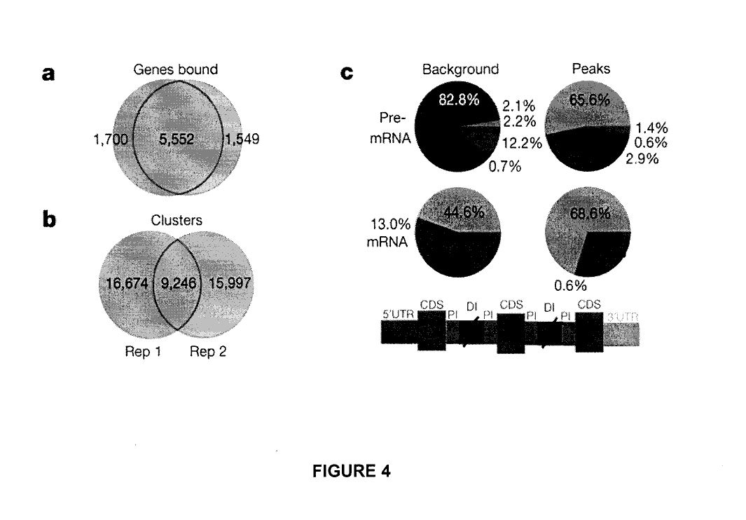

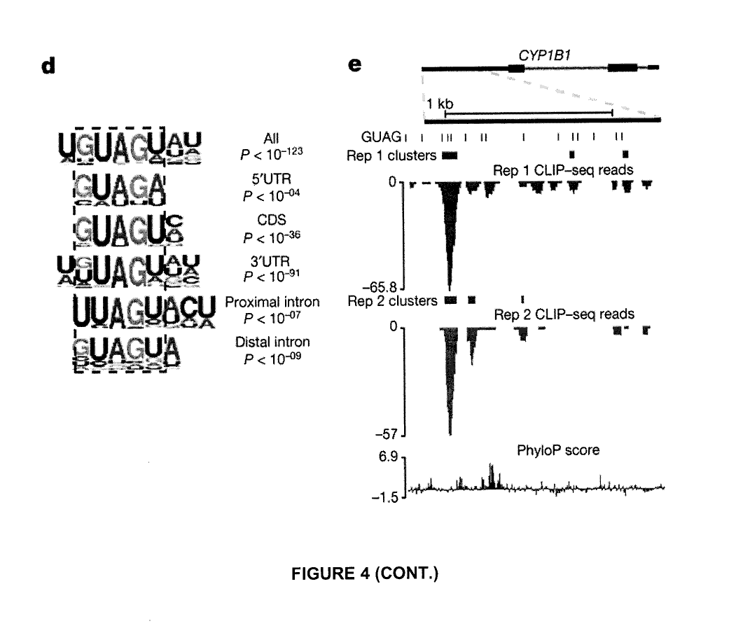

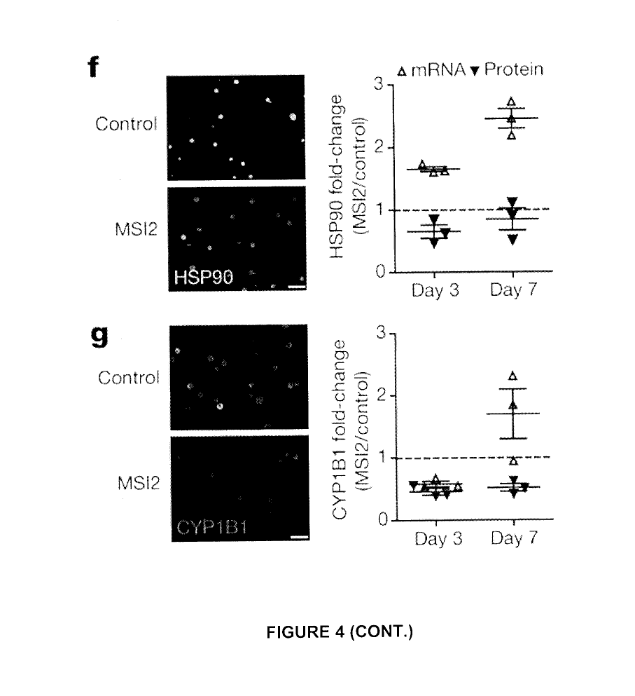

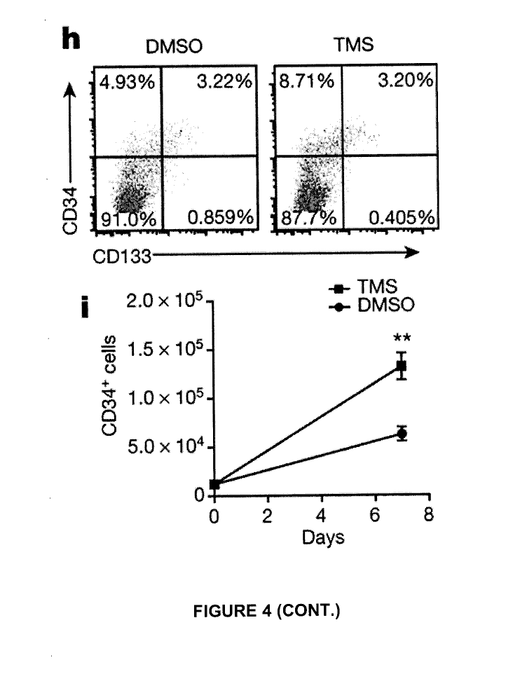

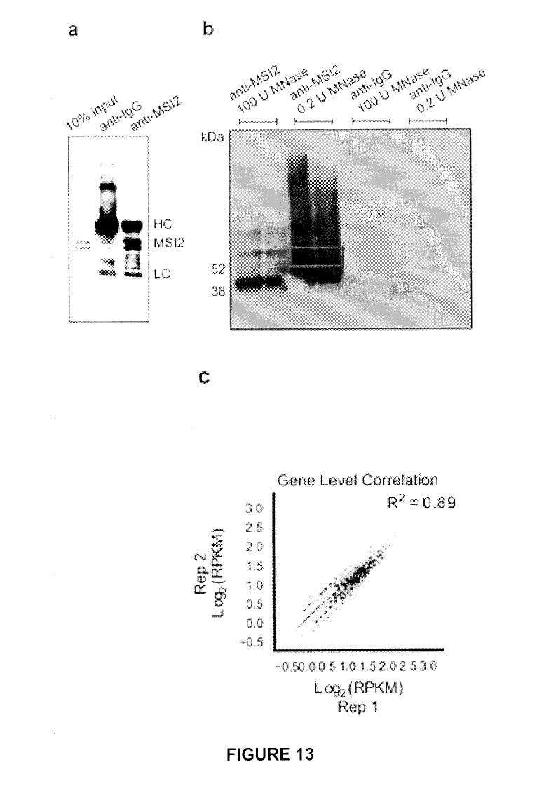

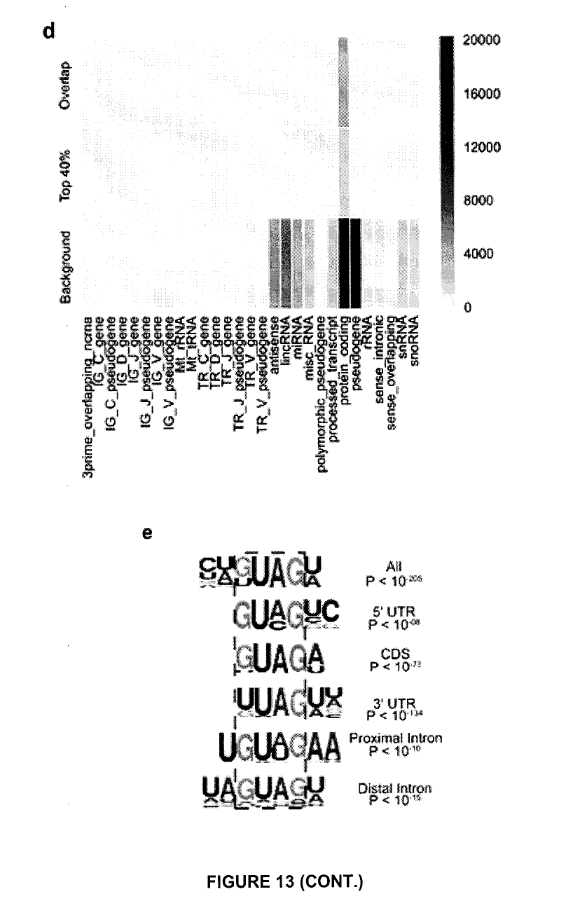

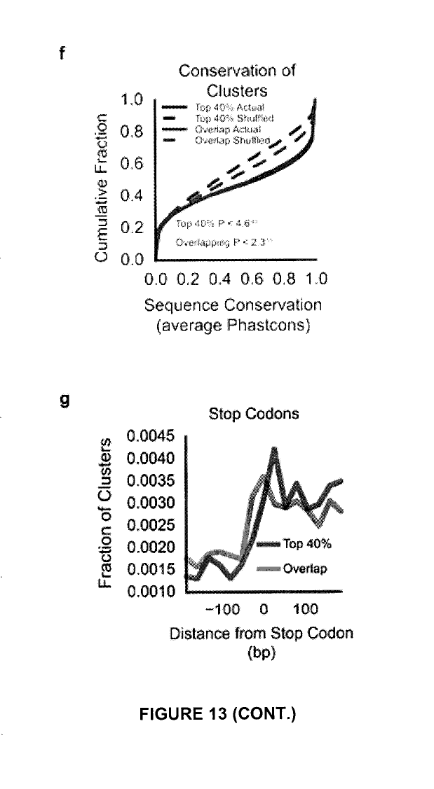

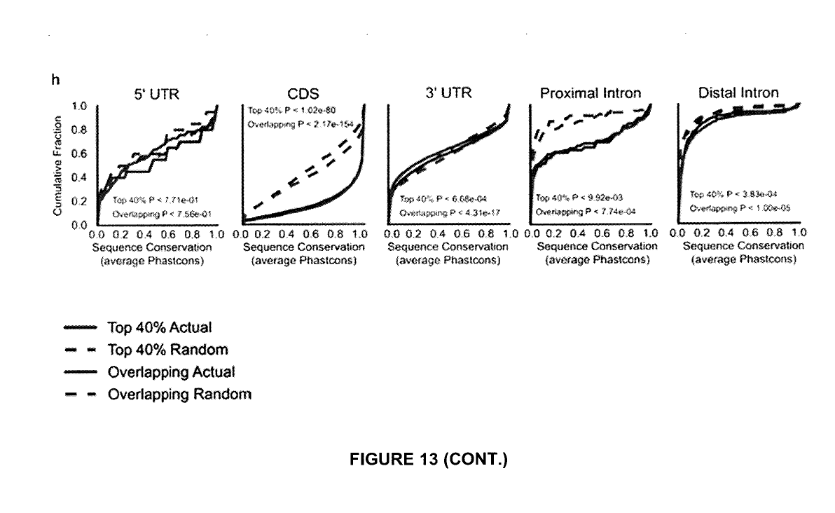

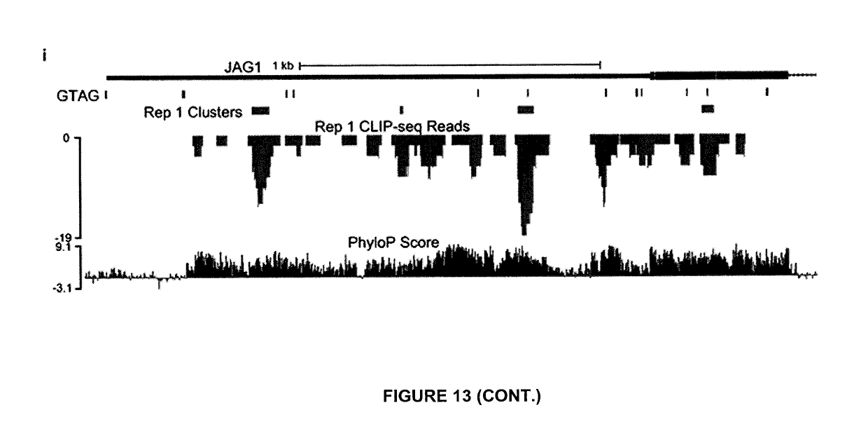

To identify key RNA targets underlying MSI2 function, we analyzed global MSI2 protein-RNA interactions using CLIP-seq (FIG. 13a, b) (Yeo et al., 2009). Mapped reads identified highly correlated gene RPKMs and enrichment of significantly overlapping clusters within >6000 protein-coding genes from replicate experiments (FIG. 13c, FIG. 4a, b). Within the top 40% of reproducible clusters, MSI2 bound predominantly to mature mRNA within their 3′ untranslated regions (3′UTRs) (FIG. 4c). Importantly, 9% of annotated protein-coding genes were reproducible MSI2 targets, compared to 0.2% of long non-coding RNAs (FIG. 13d), suggesting MSI2 controls stability or translation of coding mRNAs. Motif analyses identified a consensus pentamer (U/G)UAGU resembling the known mouse Msi1 binding sequence (Ohyama et al., 2012; Katz et al., 2014) within binding sites in all genic regions and significantly more conserved than background (FIG. 4d, FIG. 13e-h). MSI2 binding sites within Msi1 targets (Katz et al., 2014) indicates Musashi proteins may bind the same genes through 3′UTR-embedded motifs (FIG. 13i). Finally, target gene ontology analysis revealed 186 biological processes categories, among the most significant of which were electron transport, estrogen receptor signaling regulation and metabolism of small molecules, all processes known to be transcriptionally impacted by AHR signaling (Tijet et al., 2006).

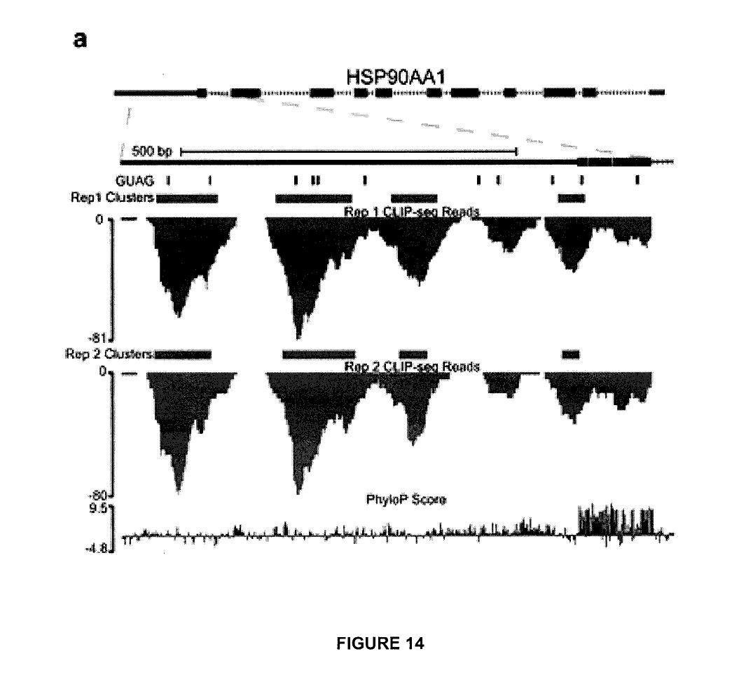

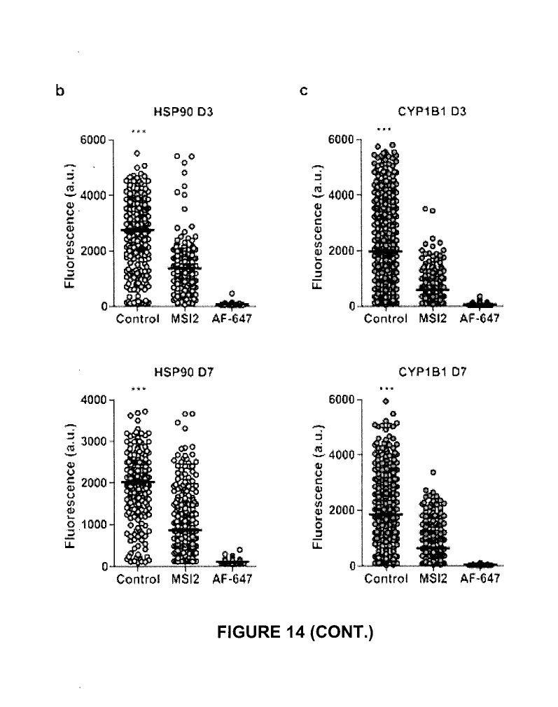

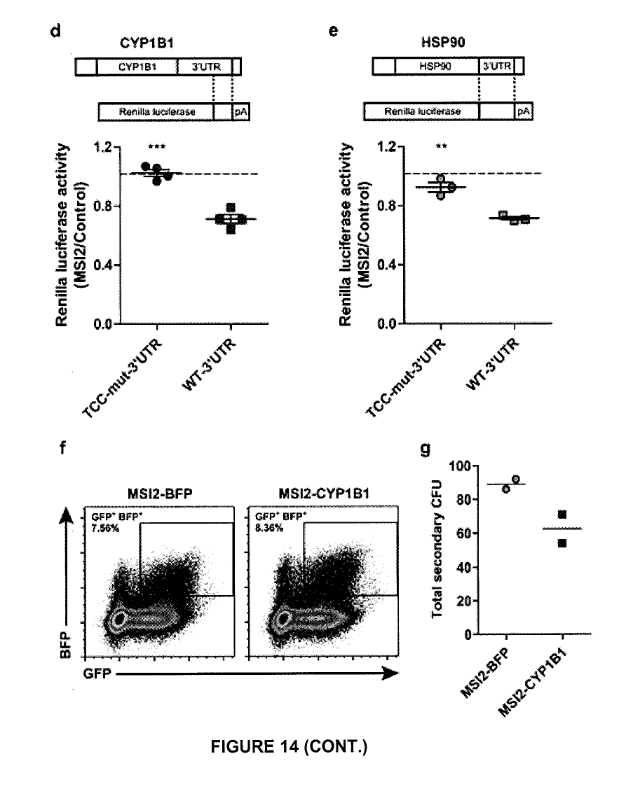

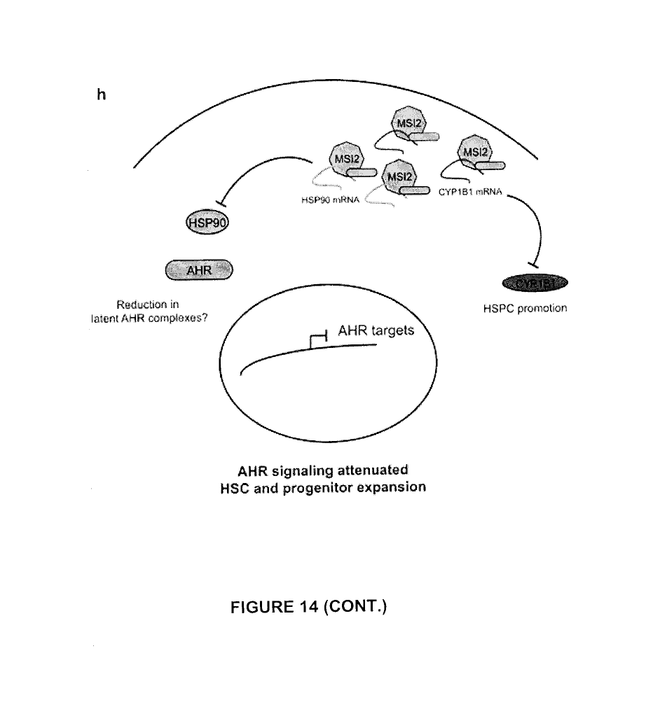

Strikingly, among the top 2% of enriched CLIP-seq targets were the 3′UTRs of heat shock protein 90 (HSP90) and CYP1B1, two AHR pathway components. Each exhibited multiple MSI2 binding motifs correlating with overlapping clusters of CLIP-seq reads (FIG. 4e, FIG. 14a). Querying MSI2's ability to post-transcriptionally regulate these genes during HSPC expansion, we looked for instances of uncoupled transcript and protein expression. HSP90 displayed uncoupling of transcript (1.6-fold up) and protein (1.6-fold down) early in culture, but at 7 days showed further upregulated transcript (2.5-fold) and variable protein levels (FIG. 4f, FIG. 14b). Since HSP90 binding of AHR is critical for its ligand-dependent transcriptional activity (Mimura et al., 2003), downregulation of HSP90 protein at the outset of HSPC culture would be expected to reduce latent AHR complex formation and attenuate AHR signaling (FIG. 3a). Indeed CYP1B1 transcript and protein displayed 2-fold reductions early in culture, consistent with decreased AHR pathway activity, however at day 7, CYP1B1 transcripts were upregulated 1.7-fold and uncoupled from 2-fold downregulated protein (FIG. 4g, FIG. 14c). To test if MSI2 directly mediates post-transcriptional repression of these targets, CYP1B1 and HSP90 3′UTR regions were coupled to luciferase. MSI2 OE induced significant reductions in luciferase signal from both reporters, an effect mitigated upon mutating the core CLIP-seq-identified UAG motifs (FIG. 14d, e). Since MSI2 OE-mediated post-transcriptional downregulation of the AHR pathway converged on CYP1B1 protein repression throughout culture, the effects of inhibiting CYP1B1 independently with (E)-2,3′,4,5′-Tetramethoxystilbene (TMS) on HSPCs were explored. During culture, TMS increased the frequency and total numbers of CD34⁺ cells by 1.5- and 2-fold respectively (FIG. 4h, i), phenocopying the effects of MSI2 OE. Lastly, co-overexpression of 3′UTRIess CYP1B1 with MSI2 decreased secondary CFU-GEMM replating efficiency (FIGS. 14f and g) suggesting that CYP1B1, while typically used to report on AHR signaling, itself enforces HSPC differentiation.

MSI2 has been identified as an important new mediator of human HSPC self-renewal and ex vivo expansion by coordinating the post-transcriptional regulation of proteins belonging to a shared self-renewal regulatory pathway (FIG. 14h).

### Example 2: CYPB1 Knockdown Increases the Number of CD34 Marked HSPCs

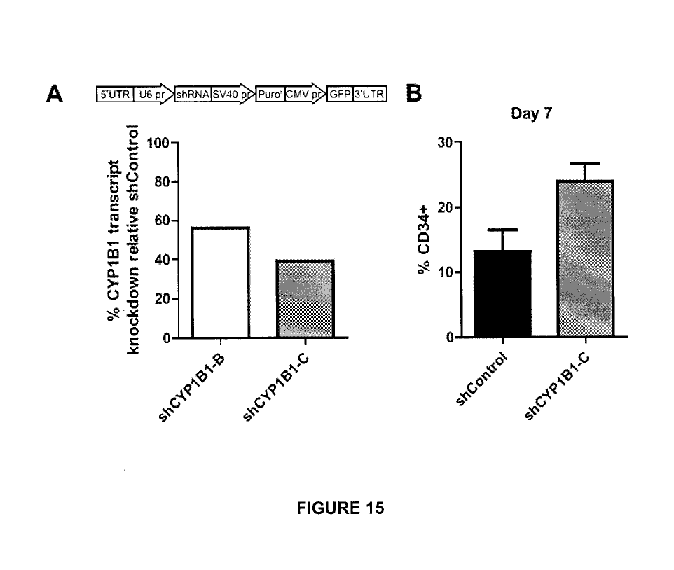

shRNA lentiviruses were tested against CYP1B1 by transducing lineage negative (Lin−) CD34+CB to read out the effects it has on HSPCs. Multiple short-hairpins were either purchased or cloned in to existing vectors (Origene, SKU:TL313605, pGFP-C-shLenti vector; shCYP1B1-C, 5′CTCCTCTTCACCAGGTATCCTGATGTGCA′3 (SEQ ID NO: 23); shCYP1B1-B, 5′TTCGAGCAGCTCAACCGCAACTTCAGCAA′3 (SEQ ID NO: 24) and tested for knockdown efficiency in the CYP1B1-expressing leukemia cell line, NB4 (FIG. 15A). Two were found to knockdown CYP1B1 transcript expression to 56% and 39% of what was observed in shControl infected cells. Subsequent experiments were then carried out with the better performing shCYP1B1-C vector in highly CD34+ enriched Lin-CB samples (>60% CD34+). Here the transcript expression was found to be at only 76% the level of what was observed in shControl cells (24% knockdown efficiency, data not shown); suggesting primary cells were more resilient to knockdown compared to cell lines. Nonetheless, it was found that even this modest reduction of CYP1B1 in CB HSPCs enhanced HSPC activity by measurement of primitive CD34 marker expression (FIG. 15B), thus phenocopying effects observed with MSI2 overexpression.

### Example 3: High Throughput Screen to Test CYP Family Inhibitors

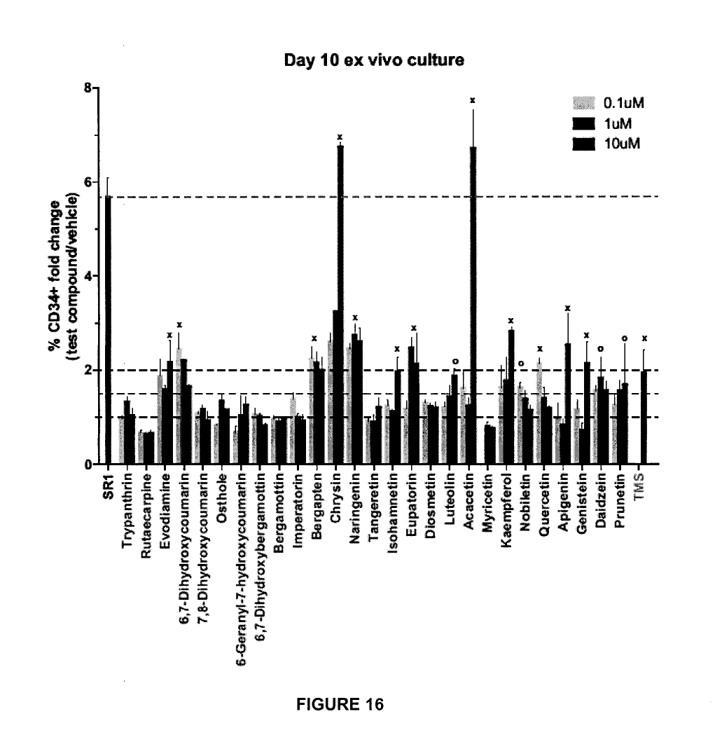

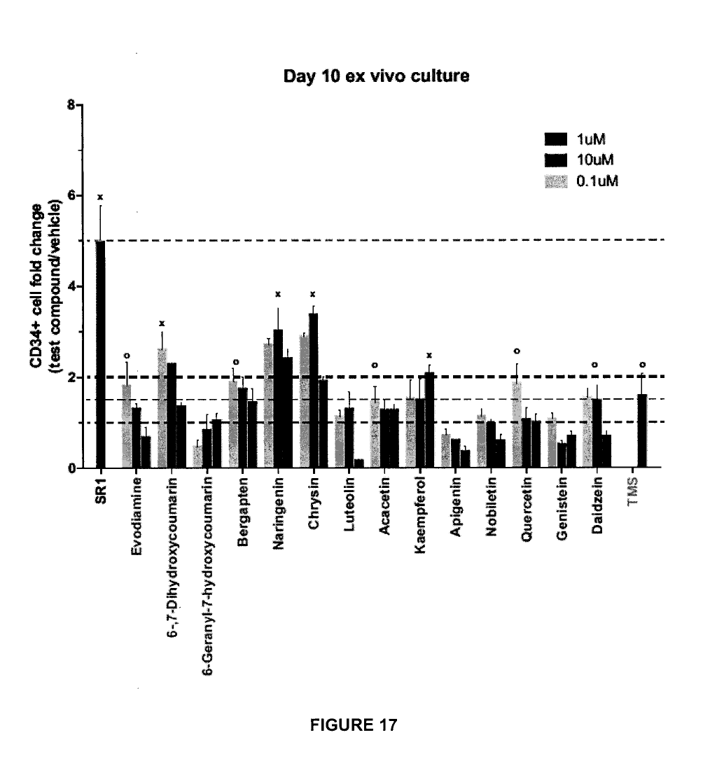

A screening approach was undertaken that tested the function of 3 alkaloid, 9 coumarin, and 17 flavonoid based compounds (27 total, Table 1). The screening approach used 96-well plates seeded with 1000 FACS-purified CD34+ cells and treatment with 3 concentrations of each compound performed in triplicate culture wells (9 wells total per compound). Cells were cultured for 10 days and resultant growth was measured for cell number and surface marker expression by automated flow cytometer (MACS Quant). Broad HSPC promoting effects were found across multiple compounds of the varying classes and concentrations tested (FIG. 16). Using TMS as a baseline for significant increase in CD34 content (>2-fold % CD34+ at day 10 relative to DMSO), and SR1 as the benchmark for best in class maintenance of CD34 marker expression, 12 compounds were found that met this cut off (“x” marked, FIG. 16) and an additional 4 were found showing a slight improvement over DMSO control (“o” marked, >1.5-fold % CD34+ at day 10 relative to DMSO, FIG. 16). Overall, when examining all compounds showing at least a 2-fold higher % CD34+ population, 52% (9/17) flavonoid compounds had a favourable response compared to 25% (2/8) coumarin and 33% (⅓) alkaloid (FIG. 16). The number of total CD34 for marked cells from a group of cultures that were either responsive or non-responsive with CYP inhibitors (FIG. 16) were examined. It was found that 9 out of 12 compounds that provided a higher frequency of CD34 marked cells and also led to improved yields of total CD34 marked cells (FIG. 17), indicating that these compounds not only maintain primitive HSC marker expression but allow for their sustained numerical expansion.

### Table 1. CYP Family Inhibitors Tested in High-Throughput Screen

TABLE 1
CYP family inhibitors tested in high-through put screen

| Class | Compound | $\frac{IC 50 {( μ M )}^{ \#}}{CYP 1 B 1}$ | $\frac{Ki {( μ M )}^{ \#}}{CYP 1 B 1}$ | Reference |
|---|---|---|---|---|
| alkaloid | Tryptanthrin | | | |
| alkaloid | Rutaecarpine | 0.055 | | 2, 4 |
| alkaloid | Evodiamine | 0.69 | | 4 |
| coumarin | 6,7-Dihydroxycoumarin | | | 1 |
| coumarin | 7,8-Dihydroxycoumarin | | | |
| coumarin | Osthole | | | |
| coumarin | 6-Geranyl-7-hydroxycoumarin | | | |
| coumarin | 6-,7-Dihydroxybergamottin | 8.89 | | 2 |
| coumarin | Imperatorin | | | |
| coumarin | Bergamottin | 7.17 | | 2 |
| coumarin | Bergapten | | | |
| flavonoid | Chrysin | | 0.016 ± 0.004 | 1 |
| flavonoid | Naringenin | 0.3* | | 1 |
| flavonoid | Targeretin | | | |
| flavonoid | Isorhamnetin | | 0.003 ± 0.0001 | 6 |
| flavonoid | Eupatorin | | 0.035 ± 0.004 | 1 |
| flavonoid | Diosmetin | <0.05* | 0.016 ± 0.089 | 1 |
| flavonoid | Luteolin | | 0.056 ± 0.011 | |
| flavonoid | Acacetin | 0.007 | 0.007 ± 0.003 | 1 |
| flavonoid | Myricellin | | 0.027 ± 0.007 | |
| flavonoid | Kaemplerol | | 0.043 ± 0.007 | |
| flavonoid | Nobiletin | | | |
| flavonoid | Quercetin | | 0.023 ± 0.01 | 2 |
| flavonoid | Apigenin | | 0.064 ± 0.010 | 3 |
| isoflavonoid | Genistein | 2.1 | | 5 |
| isoflavonoid | Daidzein | 7.9 | | 5 |
| isoflavonoid | Prunetin | | | |

*approximate values derived from graphs
^{#}values derived experimentally by EROD activity (incubation of 7-Ethoxy recrufin (7ER) with CYP enzymes
1. Toxicology. 2000 Apr. 3; 144(1-3): 31-8.
2. Molecules. 2013 Nov. 25; 18(12): 14470-95.
3. Curr Top Med Chem. 2012; 12(24): 2786-900.
4. Bioorg Med Chem Lett. 2003 Aug. 4; 13(15): 2535-8.
5. J Steroid Biochem Mol Biol. 2008 Dec; 112(4-5): 179-185.
6. Taxicol Appl Pharmacol. 20 06 May 15; 213(1): 18-26.

### Example 3: Combinatorial HSPC Promoting Effects of SR1 with TMS

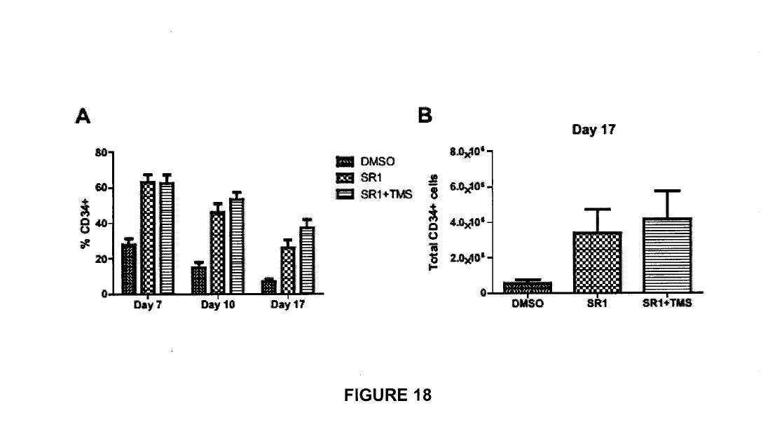

The combinatorial HSPC promoting effects of AHR antagonist SR1 with TMS (SR1+TMS) during in vitro culture was explored. It was found that particularly during extended culturing timepoints (day 17), the combined treatment of SR1+TMS led to increased frequency and number of HSPCs (FIG. 18).

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features herein before set forth, and as follows in the scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

1 Miller, P. H., Knapp, D. J. & Eaves, C. J. Heterogeneity in hematopoietic stem cell populations: implications for transplantation. Curr Opin Hematol 20, 257-264 (2013).
2 Boitano, A. E. et al. Aryl hydrocarbon receptor antagonists promote the expansion of human hematopoietic stem cells. Science 329, 1345-1348 (2010).
3 Fares, I. et al. Pyrimidoindole derivatives are agonists of human hematopoietic stem cell self-renewal. Science 345, 1509-1512 (2014).
4 Novershtern, N. et al. Densely interconnected transcriptional circuits control cell states in human hematopoiesis. Cell 144, 296-309 (2011).
5 Laurenti, E. et al. The transcriptional architecture of early human hematopoiesis identifies multilevel control of lymphoid commitment. Nat Immunol 14, 756-763 (2013).
6 Hope, K. et al. An RNAi screen identifies Msi2 and Prox1 as having opposite roles in the regulation of hematopoietic stem cell activity. Cell Stem Cell 7, 101-114 (2010).
7 de Andrés-Aguayo, L. et al. Musashi 2 is a regulator of the HSC compartment identified by a retroviral insertion screen and knockout mice. Blood 118, 554-618 (2011).
8 Park, S. M. et al. Musashi-2 controls cell fate, lineage bias, and TGF-beta signaling in HSCs. J Exp Med 211, 71-87 (2014).
9 Ohyama, T. et al. Structure of Musashil in a complex with target RNA: the role of aromatic stacking interactions. Nucleic Acids Res 40, 3218-3231 (2012).
10 Glimm, H. et al. Previously undetected human hematopoietic cell populations with short-term repopulating activity selectively engraft NOD/SCID-beta2 microglobulin-null mice. J Clin Invest 107, 199-206 (2001).
11 Cashman, J. D. & Eaves, C. J. Human growth factor-enhanced regeneration of transplantable human hematopoietic stem cells in nonobese diabetic/severe combined immunodeficient mice. Blood 93, 481-487 (1999).
12 Holyoake, T. L., Nicolini, F. E. & Eaves, C. J. Functional differences between transplantable human hematopoietic stem cells from fetal liver, cord blood, and adult marrow. Exp Hematol 27, 1418-1427 (1999).
13 Mimura, J. & Fujii-Kuriyama, Y. Functional role of AhR in the expression of toxic effects by TCDD. Biochim Biophys Acta 1619, 263-268 (2003).
14 Lo, R. & Matthews, J. High-Resolution Genome-wide Mapping of AHR and ARNT Binding Sites by ChIP-Seq. Toxicol Sci 130, 349-361 (2012).
15 Yeo, G. W. et al. An RNA code for the FOX2 splicing regulator revealed by mapping RNA-protein interactions in stem cells. Nat Struct Mol Biol 16, 130-137 (2009).
16 Katz, Y. et al. Musashi proteins are post-transcriptional regulators of the epithelial-luminal cell state. Elife 3, e03915 (2014).
17 Tijet, N. et al. Aryl hydrocarbon receptor regulates distinct dioxin-dependent and dioxin-independent gene batteries. Mol Pharmacol 69, 140-153 (2006).
18 Doulatov, S. et al. PLZF is a regulator of homeostatic and cytokine-induced myeloid development. Genes Dev 23, 2076-2163 (2009).
19 Majeti, R., Park, C. Y. & Weissman, I. L. Identification of a hierarchy of multipotent hematopoietic progenitors in human cord blood. Cell Stem Cell 1, 635-645 (2007).
20 Bagger, F. O. et al. HemaExplorer: a database of mRNA expression profiles in normal and malignant haematopoiesis. Nucleic Acids Res 41, D1034-1039 (2013).
21 Amendola, M., Venneri, M. A., Biffi, A., Vigna, E. & Naldini, L. Coordinate dual-gene transgenesis by lentiviral vectors carrying synthetic bidirectional promoters. Nat Biotechnol 23, 108-116 (2005).
22 van Galen, P. et al. The unfolded protein response governs integrity of the haematopoietic stem-cell pool during stress. Nature 510, 268-272 (2014).
23 Lechman, E. R. et al. Attenuation of miR-126 activity expands HSC in vivo without exhaustion. Cell Stem Cell 11, 799-811 (2012).
24 Carow, C. E., Hangoc, G. & Broxmeyer, H. E. Human multipotential progenitor cells (CFU-GEMM) have extensive replating capacity for secondary CFU-GEMM: an effect enhanced by cord blood plasma. Blood 81, 942-949 (1993).
25 Milyaysky, M. et al. A distinctive DNA damage response in human hematopoietic stem cells reveals an apoptosis-independent role for p53 in self-renewal. Cell Stem Cell 7, 186-197 (2010).
26 Janky, R. et al. iRegulon: from a gene list to a gene regulatory network using large motif and track collections. PLoS Comput Biol 10, e1003731 (2014).
27 Subramanian, A. et al. Gene set enrichment analysis: a knowledge-based approach for interpreting genome-wide expression profiles. Proc Natl Acad Sci USA 102, 15545-15550 (2005).
28 Kwon, A. T., Arenillas, D. J., Worsley Hunt, R. & Wasserman, W. W. oPOSSUM-3: advanced analysis of regulatory motif overrepresentation across genes or ChIP-Seq datasets. G3 (Bethesda) 2, 987-1002 (2012).
29 Colvin, G. A. et al. Murine marrow cellularity and the concept of stem cell competition: geographic and quantitative determinants in stem cell biology. Leukemia 18, 575-583 (2004).
30 Hu, Y. & Smyth, G. K. ELDA: extreme limiting dilution analysis for comparing depleted and enriched populations in stem cell and other assays. J Immunol Methods 347, 70-78 (2009).
31 Langmead, B., Trapnell, C., Pop, M. & Salzberg, S. L. Ultrafast and memory-efficient alignment of short DNA sequences to the human genome. Genome Biol 10, R25 (2009).
32 Dobin, A. et al. STAR: ultrafast universal RNA-seq aligner. Bioinformatics 29, 15-21 (2013).
33 Darnell, R. CLIP (cross-linking and immunoprecipitation) identification of RNAs bound by a specific protein. Cold Spring Harb Protoc 2012, 1146-1160 (2012).
34 Lovci, M. T. et al. Rbfox proteins regulate alternative mRNA splicing through evolutionarily conserved RNA bridges. Nat Struct Mol Biol 20, 1434-1442 (2013).
35 Quinlan, A. R. & Hall, I. M. BEDTools: a flexible suite of utilities for comparing genomic features. Bioinformatics 26, 841-842 (2010).
36 Dale, R. K., Pedersen, B. S. & Quinlan, A. R. Pybedtools: a flexible Python library for manipulating genomic datasets and annotations. Bioinformatics 27, 3423-3424 (2011).
37 Liao, Y., Smyth, G. K. & Shi, W. feature Counts: an efficient general purpose program for assigning sequence reads to genomic features. Bioinformatics 30, 923-930 (2014).
38 Liu, J., Sridhar, J and Foroozesh, M. Cytochrome P450 Family 1 Inhibitors and Structure-Activity Relationships. Molecules 2013, 18, 14470-14495.
39 Doostdar, H., Burke, M. Danny, and Mayer, Richard T. Bioflavonoids: selective substrates and inhibitors for cytochrome P450 CYP1A and CYP1B1. Toxicology 144 (2000) 31-38
40 Xu, S., et al., Preferential expression of cytochrome CYP CYP2R1 but not CYP1B1 in human cord blood hematopoietic stem and progenitor cells. Acta Pharm Sin B, 2014. 4(6): p. 464-9.
41 Fares, I., et al., Pyrimidoindole derivatives are agonists of human hematopoietic stem cell self-renewal. Science. 2014 Sep. 19; 345(6203): 1509-1512.
42 Gundry et al., Highly Efficient Genome Editing of Murine and Human Hematopoietic Progenitor Cells by CRISPR/Cas9. Cell Reports, 2016 Oct. 25,17(5):1453-1461.
43 Krosl et al., In vitro expansion of hematopoietic stem cells by recombinant TAT-HOXB4 protein. Nat Med 2003, 2003 November; 9(11):1428-32.
44 Bolhassani et al., In vitro and in vivo delivery of therapeutic proteins using cell penetrating peptides. Peptides 2017, January; 87:50-63.

1. A method of increasing the self-renewal and/or expansion of hematopoietic stem and progenitor cells (HSPCs), the method comprising:
inhibiting the activity and/or expression of cytochrome P450 1B1 (CYP1B1), or
increasing the activity and/or expression of Musashi-2 (MSI2),
in a population of one or more HSPCs.

2. The method of claim 1, wherein inhibiting the activity and/or expression of CYP1B1 or increasing the activity and/or expression of MSI2 increases the frequency and/or number of HSPCs in the population of cells relative to a control population of cells.

3. The method of claim 1, wherein inhibiting the activity and/or expression of CYP1B1 comprises contacting the HSPCs with a CYP1B1 inhibitor or increasing the activity and/or expression of MSI2 comprises contacting the HSPCs with a MSI2 activator.

4. The method of claim 3, wherein the CYP1B1 inhibitor is a stilbenoid, flavonoid, coumarin, alkaloid, a siRNA molecule or a shRNA molecule that decreases expression of CYP1B1.

5. (canceled)

6. The method of claim 4, wherein:
the stilbenoid is 2,4,3′,5′-Tetramethoxystilbene (2,4,3′,5′-TMS), 2,4,2′,6′-Tetramethoxystilbene (2,4,2′,6′-TMS) or (E)-2,3′,4,5′-Tetramethoxystilbene (2,3′,4,5′-TMS),
the flavonoid is 3,5,7-Trihydroxy-2-(4-hydroxyphenyl)-4H-chromen-4-one (Kaempferol), 5,7-Dihydroxy-2-phenyl-4H-chromen-4-one (Chrysin), 5,7-Dihydroxy-2-(4-hydroxyphenyl)chroman-4-one (Naringenin), 3,6,7-Trihydroxy-2-(4-hydroxy-3-methoxyphenyl)-4H-chromen-4-one (Isohamnetin), 3′,5-Dihydroxy-4′,6,7-trimethoxyflavone (Eupatorin), 2-(3,4-Dihydroxyphenyl)-5,7-dihydroxy-4-chromenone (Luteolin), 5,7-dihydroxy-2-(4-methoxyphenyl)-4H-1-benzopyran-4-one (Acacetin), 2-(3,4-Dimethoxyphenyl)-5,6,7,8-tetramethoxychromen-4-one (Nobiletin), 2-(3,4-dihydroxyphenyl)-3,5,7-trihydroxy-4H-chromen-4-one (Quercetin), 5,7-Dihydroxy-2-(4-hydroxyphenyl)-4H-1-benzopyran-4-one (Apigenin), 5,7-Dihydroxy-3-(4-hydroxyphenyl)chromen-4-one (Genistein), 7-Hydroxy-3-(4-hydroxyphenyl) chromen-4-one (Daidzein) or 5-hydroxy-3-(4-hydroxyphenyl)-7-methoxychromen-4-one (Prunetin),
the coumarin is 6-,7-dihydroxycoumarin or 5-methoxypsoralen (bergapten),
the alkaloid is evodiamine, or
the shRNA molecule comprises a sequence with at least 80% sequence identity to SEQ ID NO: 23 or SEQ ID NO: 24.

7.-13. (canceled)

14. The method of claim 3, wherein the CYP1B1 inhibitor is evodiamine, 6,7-dihydroxycoumarin, bergapten, chrysin, naringenin, isohamnetin, eupatorin, liteloin, acacetin, kaempferol, nobiletin, quercetin, apigenin, genistein, daidzein, prunetin or TMS.

15.-17. (canceled)

18. The method of claim 1, further comprising contacting the HSPCs with SR1 or a pyrimidoindole derivative.

19.-21. (canceled)

22. The method of claim 3, wherein the MSI2 activator is:
a nucleic acid molecule encoding MSI2 or a functional fragment thereof, or
a MSI2 protein or a functional fragment thereof, and optionally wherein the MSI2 protein or the functional fragment thereof is conjugated to a protein transfection reagent.

23. (canceled)

24. The method of claim 1, wherein the HSPCs are CD34+ cells.

25. The method of claim 1, wherein the HSPCs are from cord blood, umbilical cord, mobilized peripheral blood or bone marrow.

26. The method of claim 1, wherein the HSPCs are in vivo, ex vivo or in vitro.

27. The method of claim 1, further comprising transplanting the expanded HSPCs to a subject in need thereof.

28. The method of claim 27, wherein the HSPCs are autologous HSPCs or allogenic HSPCs.

29. The method of claim 27, wherein the subject has a hematopoietic disorder, malignancy, autoimmune disease and/or immunodeficiency.

30. The method of claim 29, wherein the hematopoietic disorder is thalassemia, sickle cell anemia, aplastic anemia or fanconi anemia.

31. The method of claim 29, wherein the malignancy is a cancer of the blood or bone marrow.

32. The method of claim 31, wherein the cancer of the blood or bone marrow is non-Hodgkin's lymphoma, Hodgkin's lymphoma, multiple myeloma or leukemia.

33. The method of claim 27, wherein the subject has received chemotherapy and/or radiation.

34.-41. (canceled)

42. A composition comprising one or more hematopoietic stem and progenitor cells (HSPCs) and a cytochrome P450 1B1 (CYP1B1) inhibitor or a Musashi-2 (MSI2) activator.

43.-45. (canceled)

46. A composition comprising a CYP1B1 inhibitor or a MSI2 activator, and an agent that induces hematopoiesis, wherein the agent that induces hematopoiesis is SR1 or a pyrimidoindole derivative.

47.-57. (canceled)
